SEQUENCE LISTING (4 sequences)

SEQ ID NO: 1 - protein, 37 aa, Artificial sequence
  85B
MFQDAYNAAGGHNAVFGSGATNFSLLKQAGDVEENPG

SEQ ID NO: 2 - protein, 59 aa, Artificial sequence
  85B-C5
MFQDAYNAAGGHNAVFGSGATNFSLLKQAGDVEENPGPAAQAAVVRFQEAANKQKQELD

SEQ ID NO: 3 - DNA, 1101 bp, Artificial sequence
  DNA sequences of the CMV promoter, 85B epitope, peptide 2A, and BGH polyadenylation
  n (20)..(20): deoxythymidine
  n (21)..(21): deoxythymidine
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgttccaggacgcctacaacgctgctggaggacacaacgccgtgttcggcagcggagctaccaacttttccctgctgaagcaggctggcgatgtggaggagaaccccggataagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 4 - DNA, 1179 bp, Artificial sequence
  DNA sequences of the CMV promoter, 85B epitope, peptide 2A, AIP-C5 peptide, and BGH polyadenylation
  n (20)..(20): deoxythymidine
  n (21)..(21): deoxythymidine
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcgatatcggatccgccaccatgttccaggacgcctacaacgctgctggaggacacaacgccgtgttcggcagcggagctaccaacttttccctgctgaagcaggctggcgatgtggaggagaacccaggacctgctgctcaggctgctgtggtgaggtttcaggaggccgctaacaagcagaagcaggagctggactaagcggccgcgatatctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

(19) United States
(12) Patent Application Publication
(10) Pub. No.: US 2024/0366741 A1
(43) Pub. Date: Nov. 7, 2024

(54) NOVEL VACCINE FORMULATIONS FOR MYCOBACTERIUM TUBERCULOSIS AND USE OF THEREOF

(71) Applicants: Purdue Research Foundation, West Lafayette, IN (US); HOUSTON METHODIST RESEARCH INSTITUTE, Sugar Land, TX (US)

(72) Inventors: Suresh Kumar Mittal, West Lafayette, IN (US); Chinnaswamy Jagannath, Sugar Land, TX (US)

(73) Assignees: Purdue Research Foundation, West Lafayette, IN (US); HOUSTON METHODIST RESEARCH INSTITUTE, Sugar Land, TX (US)

(21) Appl. No.: 18/550,036

(22) PCT Filed: Mar. 8, 2022
(86) PCT No.: PCT/US2022/019222

§ 371 Date: Sep. 11, 2023

Related U.S. Application Data
(60) Provisional application No. 63/160,035, filed on Mar. 12, 2021.

(51) Int. Cl. A61K 39/04; A61K 39/00; A61P 31/06; C12N 15/86

(52) U.S. Cl. CPC A61K 39/04; A61P 31/06; C12N 15/86; A61K 2039/53; A61K 2039/543; A61K 2039/552; A61K 2039/57; C12N 2710/10043; C12N 2710/10343

(57) ABSTRACT

The present invention discloses a recombinant adenovirus vector of a replication-defective human adenovirus (HAdv^{85C5}) or a bovine adenovirus (BAdv^{85C5}) comprising a recombinant adenovirus vector having a heterologous DNA segment encoding mycobacterial Ag85B-p25 epitope (SEQ ID NO: 1), mycobacterial Ag85B-p25 epitope fusion of autophagy-inducing peptide-C5 (SEQ ID NO: 2), or a substantially homologous functional fragment thereof. The vector, having a heterologous DNA segment of SEQ ID NO: 3, SEQ ID NO: 4, or a substantially homologous functional fragment thereof, is an effective vaccine for therapeutically or prophylactically immunizing a subject for protection of infections by various microorganisms, especially Mycobacterium tuberculosis (Mtb), which causes the widespread tuberculosis. Methods of uses and pharmaceutical composition matters are within the scope of this disclosure.

### CROSS-REFERENCE TO RELATED APPLICATIONS

The present U.S. patent application relates to and claims the priority benefit of U.S. Provisional Patent Application Ser. No. 63/160,035, filed Mar. 12, 2021, the contents of which are hereby incorporated by reference in its entirety into this disclosure.

### GOVERNMENT SUPPORT CLAUSE

This invention was made with government support under AI122070 and AI138587 awarded by the National Institutes of Health. The government has certain rights in the invention.

### STATEMENT OF SEQUENCE LISTING

A computer-readable form (CRF) of the Sequence Listing is submitted concurrently with this application. The file, entitled 68665-02_Seq_Listing_ST25_txt, having a size of 5 kb, is generated on Feb. 22, 2022. Applicant states that the content of the computer-readable form is the same and the information recorded in computer readable form is identical to the written sequence listing.

### TECHNICAL FIELD

The present disclosure generally relates to a recombinant adenovirus vector of a replication-defective human adenovirus (HAdv^{85C5}) or a bovine adenovirus (BAdv^{85C5}) comprising a heterologous DNA segment encoding mycobacterial Ag85B-p25 epitope (SEQ ID NO: 1) or mycobacterial Ag85B-p25 epitope fusion of autophagy-inducing peptide-C5 (SEQ ID NO: 2). The vector is an effective vaccine for protection of infections by various microorganisms, especially Mycobacterium tuberculosis (Mtb), which causes the widespread tuberculosis.

### BACKGROUND

This section introduces aspects that may help facilitate a better understanding of the disclosure. Accordingly, these statements are to be read in this light and are not to be understood as admissions about what is or is not prior art.

Mycobacterium tuberculosis (Mtb) is a leading cause of mortality due to tuberculosis and kills about 1.5 million people each year with 8 million new cases. Bacillus Calmette-Guérin (BCG), a live attenuated vaccine is widely used for primary immunization of children around the world; although, it protects mostly against extra pulmonary tuberculosis and shows a variable protection against pulmonary disease ranging from 0-80%. Variation in the genetics of the population, lack of RD1 operon in BCG, and pre-exposure to environmental mycobacteria together are thought to influence the efficacy of BCG.

We sought to improve BCG- and Mtb-derived vaccines and identified that the BCG vaccine sequesters within immature phagosomes of the antigen-presenting cells (APCs) similar to Mtb localization in macrophages (MΦs) and dendritic cells (DCs) [APCs], and thus are poorly delivered to the lysosomes (Singh et al., 2006; Singh et al., 2011). We showed that sequestration of BCG in immature phagosomes led to a decreased presentation of BCG-derived Ag85B-p25 epitope to CD4 T cells in mice and human MΦs (Jagannath et al., 2009; Singh et al., 2006). Next, we discovered that an induction of autophagy in APCs with rapamycin enhanced the delivery of both Mtb and BCG to lysosomes, thereby increasing antigen presentation to CD4 T cells in vitro (Jagannath et al., 2009) We then demonstrated that a second-generation recombinant BCG vaccine over-expressing Ag85B (BCG^{85B}) induced autophagy in APCs and was more effective than BCG in mice against tuberculosis (Jagannath et al., 2009). Besides, the autophagy-inducing drug, rapamycin, enhanced the efficacy of the BCG vaccine in mice against tuberculosis (Bakhru, 2012; Bakhru et al., 2013). A third generation BCG^{85BC5} vaccine expressed a TLR2-activating and autophagy-inducing peptide C5 (AIP-C5) from Mtb CFP10 protein and was even more potent than BCG^{85B} against tuberculosis in mice (Khan et al., 2019). These studies confirmed that autophagy induction boosts vaccine efficacy. Indeed, an autophagy-inducing recombinant BCG vaccine has entered human clinical trials (Gengenbacher et al., 2016).

Although, millions of children are already receiving BCG vaccination at birth, but most remain susceptible to lung tuberculosis. Thus, childhood tuberculosis is quite common in developing countries. Despite the ability of BCG to protect against extra pulmonary infections, tuberculosis meningitis continues to be a problem in children (Ramzan et al., 2009). Since the main portal of entry for Mtb is the respiratory route, strengthening the lung immune response seems to be a rational approach to prevent both pulmonary and extra-pulmonary tuberculosis. Indeed, many studies document that booster vaccines using viral vectors or subunit vaccines augment the efficacy of BCG in mice against tuberculosis; although, the route of their vaccination seems to affect their efficacy. For example, the intramuscular vaccination with human adenovirus (Adv) type 5 (HAdv5) vectors expressing various Mtb antigens protected mice against tuberculosis to varying degrees (Santosuosso et al., 2006; Xing et al., 2009; Hoft et al., 2012; van Zyl-Smit et al., 2017). HAdv5 vector-based tuberculosis vaccines elicit potent CD4 and CD8 T cells responses among human adults despite pre-existing adenoviral vector immunity (Ahi et al., 2011; Fausther-Bovendo and Kobinger, 2014). Others showed that a recombinant influenza virus vector expressing Ag85B-p25 epitope protected mice reducing the lung burden of Mtb by <0.5-log 10 compared to unvaccinated controls; although, a booster vaccination was not evaluated (Florido et al., 2015). The latter vaccine also induced lung resident memory T cells (T_{RM}) protecting adult mice from lung tuberculosis, even when circulating T cells were depleted (Florido et al., 2018). Paradoxically, the aerosol or intramuscular inoculation of BCG-vaccinated adult rhesus macaques with HAdv5 vectors expressing multiple Mtb antigens did not protect them from tuberculosis (Darrah et al., 2019). Importantly, immunization of BCG-vaccinated children with a modified vaccinia virus vector-expressing-Ag85A (MVA85A) was not effective in preventing tuberculosis (Tameris et al., 2013). There are still unmet clinical needs of effective protection and treatment for tuberculosis.

### BRIEF DESCRIPTION OF DRAWINGS

FIG. 1A: Diagrammatic representation of the gene cassette of the Ag85B-p25 epitope of Mycobacterium tuberculosis without or with the autophagy-inducing peptide C5 (AIP-C5) and the resultant bovine adenovirus type 3 (BAdv) or human adenovirus type 5 (HAdv) vectors. The two peptides were separated by an Ala-Ala-Ala linker. The gene cassette was under the control of the cytomegalovirus (CMV) promoter and the bovine growth hormone (BGH) polyadenylation (PA) signal. The drawings are not up to the scale. 85, Ag85B-p25 epitope; C5, AIP-C5; LTR & RTR, the left and right terminal repeats; ΔE1, deletion of the early region 1; ΔE3, deletion of the early region 3. FIG. 1B: HAd5 transcription map. FIG. 1C: HAd-ΔE1E3 transcription map showing E1 and E3 deletions. FIG. 1D: BAd3 transcription map. FIG. 1E: BAd-ΔE1E3 transcription map showing E1 and E3 deletions.

FIGS. 2A-2F: Replication-defective BAdv^{85C5} (BAdv vector expressing 85B epitope+AIP-C5) vaccine induces a unique transcriptome in mouse dendritic cells compared to HAdv^{85C5} (HAdv vector expressing 85B epitope+AIP-C5) vaccine. FIG. 2A: Heat maps of differentially expressed genes (DEGs) among DCs infected with BAdv- or HAdv-derived vaccines or vectors are shown. FIGS. 2B-2E: Gene expression analysis using Clusterprofiler indicated as Gene Ontology (GO), Cellular Components (CC) and Biological Processes (BP) followed by KEGG (Kyoto Encyclopedia of Genes and Genomes) profiles. FIG. 2F: Heatmaps of transcripts derived from RNAseq expressed as FPKMs (Fragments per kilo base per million mapped reads; n=2); BAdv^{85C5} vaccine upregulated genes involved in antigen processing and presentation are reproduced and those selectively enriched are blue highlighted (p<0.0001, n=2).

FIGS. 3A-3C: Replication-defective BAdv^{85C5} vaccine is rapidly internalized by mouse dendritic cells (DCs) and induces a unique transcriptome. CD11c⁺ DCs purified from bone marrows of wt-C57BL/6 mice infected with the BAdv^{85C5} vaccine or control vector (10⁷ PFU/10⁶ DCs), followed by antibody staining for intracellular localization using confocal microscopy at 4 h post-infection. FIGS. 3A-3C: Cy3-labeled-BAdv vector or BAdv^{85C5} vaccine infected DCs were incubated for a 4 h infection, followed by fixation and staining using specific antibodies to (FIG. 3A) microtubule associate light chain 3 (LC3) autophagosome marker; (FIG. 3B) Lysosome-associated membrane proten1 (LAMP1), (FIG. 3C) Galectin3 (Lgals-3). Panels show Cy3 (red), Alexfluor488 (green) and merged images with or without DAPI nuclear stain. Colocalization (inset) analyzed using confocal microscopy and Nikon N90 microscope with Metaview software. Cytosolic (white arrow) and nuclear localization (yellow arrow) of Cy3-labeled vector or vaccines are indicated. Bar graphs to the right show percent virus containing endosomes colocalizing with antibodies calculated per DC and averaged for 20 DCs per experiments done twice. *p<0.01 t test.

FIGS. 4A-4G: Replication-defective BAdv^{85C5} vaccine enhances autophagy-dependent and independent antigen presentation in mouse dendritic cells (DCs) and human macrophages (MΦs). FIG. 4A: wt-C57BL/6- or ATG7KO-C57BL/6-derived DCs were infected with vaccines or control vectors followed by overlay using BB7 CD4 T cells specific for the Ag85B-p25 epitope in the context of MHC-II. IL-2 in supernatants at 18 h post-infection was determined using sandwich ELISA. FIG. 4B: Antigen presentation in vaccine or vector infected mouse MΦs is shown. FIGS. 4C-4D: Human PBMC-derived CD14⁺ MΦs were infected with vaccines or control vectors followed by an overlay with F9A6 T cells specific for an Ag85B epitope in the context of human HLA-DR1 for antigen presentation. MΦs were untreated or treated with 3-methyladenine (50 μM) before infection to inhibit autophagy. FIG. 4E: Time-dependent antigen presentation by human MΦs infected with BAdv or HAdv derived vaccines. Horizontal line shows IL-2 levels of T cells incubated over naïve MΦs. FIGS. 4F-4G: CD14⁺ mouse bone marrow-derived MΦs were infected with 10⁶ PFU of BAdv vector, BAdv⁸⁵ or BAdv^{85C5} followed by coinfection with Mtb (Erdman strain; MOI=1) and incubation. MΦ lysates were plated for CFU counts at indicated time points.

FIGS. 5A-5G: Replication-defective BAdv^{85C5} vaccine enhances galectin and cathepsin-dependent antigen presentation in mouse dendritic cells (DCs) and human macrophage (MΦs). C57BL/6 mouse derived DCs were infected with vaccines or control vectors, washed and processed. FIG. 5A: At 18 h post infection lysates collected in Trizol were used for qPCR using primers for endosome trafficking genes enriched during transcriptomics (FIG. 2) (*≥1.4-fold increase in mRNA expression, n=2, 2 experiments). FIG. 5B: At 18 h post infection lysates in anti-protease buffer analyzed using specific antibodies and Wes Simple blot protocol. Densitometry panels shown. FIGS. 5C-5D: DCs and MΦs were subjected to siRNA vs. (Lgals-3, L-gals-8) or scrambled controls and at 18 h, infected using vaccines or vectors for 4 h. Washed DCs were overlaid using BB7 CD4 T cells and 18 h later, supernatants assayed for IL-2 using sandwich ELISA (triplicate wells of DCs per group and 2 independent experiments). FIG. 5E: Lysates collected at 18 h post infection were used for qPCR using primers for cathepsins enriched during transcriptomics (FIG. 2) (*≥1.4-fold increase in mRNA expression, n=2, 2 experiments). FIG. 5F: Lysates collected at 18 h post infection were analyzed using specific antibodies and Wes Simple blot protocol. FIG. 5G: wt-DCs or ATG7KO-DCs DCs were left untreated or treated with cathepsin inhibitors as indicated, followed by vaccine or vector infection and antigen presentation. Abbrev: N-acetyl-L-leucyl-L-leucyl-L-methional (NALLM), calpeptin; E64, each at 30 μM; triplicate wells of DCs per group and 2 independent experiments; *p<0.01, **p<0.006, 1-way ANOVA with Tukey's post-test.

FIGS. 6A-6D: BAdv^{85C5} mucosal vaccine protects mice against aerosolized Mycobacterium tuberculosis either alone or as a booster following BCG vaccine. FIG. 6A: 4-6-week-old female or male C57BL/6 mice were nasally instilled using once dose each of vaccines or vectors at 10⁷ PFU After 3 days, mice were sacrificed and bronchoalveolar lavage (BAL) cells and lung tissue suspended in Trizol and subjected to qPCR for gene expression (*≥1.4-fold increase in mRNA expression, n=2, 2 experiments) (n=2 for each group; 2 experiments). FIG. 6B: 4-6-week-old female or male C57BL/6 mice were mock-inoculated or vaccinated subcutaneously with BCG, and at 7 days post-immunization, animals received a single intranasal booster vaccination with 10⁷ PFU of BAdv85, BAdv^{85C5} or BAdv vector control. Three weeks later, mice were aerosol challenged with Mtb Erdman using a Glas-Col inhalation apparatus. Four 4 weeks later mice were sacrificed for Mtb counts followed by analysis of the lungs and spleen for T cell profiles (FIGS. 7 & 8). FIG. 6C: BAdv^{85C5} reduces the lungs and spleen burden of Mtb (5 mice per group for CFU; ***, p<0.004; **, p<0.007; 2-way ANOVA with Dunett's post-test). FIG. 6D: Lungs and spleen of mice collected post-Mtb challenge were stained using Ag85-p25 epitope-specific MHC-II tetramer (NIH tetramer core facility, Emory Univ, USA) and analyzed using flow cytometry (n=3 from the same group of 4M+8F mice; ****, p<0.001; ordinary 1-way ANOVA). Arrowheads indicate BAdv^{85C5} induced T cell expansion.

FIGS. 7A-7H: BAdv^{85C5} booster induces stronger cytokine positive T cells and effector and memory T cells in BCG vaccinated mice following Mtb challenge. FIGS. 7A-7D: Lungs and spleens of mice vaccinated and challenged as in FIG. 4 were stained for CD4 and CD8 T cells expressing IFN-γ and IL-2 and analyzed using flow cytometry (****, p<0.001; ordinary 1-way ANOVA). Arrowheads indicate BAdv^{85C5}-induced T cell expansion. FIGS. 7E-7H: The lungs and spleen of mice vaccinated and challenged as in FIG. 6 were stained for CD4 and CD8 effector (T_{EM}) and central memory T cells (T_{CM}) followed by flow cytometric analysis (*p<0.01. ***, ****p<0.001, ordinary 1-way ANOVA). Arrows indicate BAdv^{85C5}-induced T cell expansion.

FIGS. 8A-8C: BAdv^{85C5} booster induces expansion of the lung and spleen resident memory T cells (TRMs) in BCG-vaccinated mice before and after Mtb challenge. FIG. 8A: The lungs (FIG. 8B) and spleens (FIG. 8C) of mice vaccinated and challenged were stained for TRMs on day 21 for post vaccination (vax) and day 60 post Mtb challenge and analyzed using flow cytometry (*, p<0.01, **p<0.009, ordinary 1-way ANOVA).

FIG. 9A: qPCR validation of Antigen 85B derived p25 and CFP110 derived C5 gene (proprietary) genes in BAdv^{85C5} infected DCs compared to HAdv^{85C5} infected DCs. FIG. 9B: Diagrammatic representation of the gene cassette of the Ag85B-p25 epitope of Mycobacterium tuberculosis without or with the autophagy-inducing peptide C5 (AIP-C5) and the resultant HAdv vectors.

FIGS. 10A-10B: qPCR validation of genes enriched in BAdv^{85C5} infected DCs (FIG. 10A) compared to HAdv^{85C5} infected DCs (FIG. 10B) (*≥1.4-fold increase over vector controls).

### BRIEF DESCRIPTION OF SEQUENCE LISTINGS

Protein/Peptide sequence 85B: (Met+AG85B CD4 EPITOPE+GSG linker+P2A (self-cleavage peptide):

(SEQ ID NO: 1)
MFQDAYNAAGGHNAVFGSGATNFSLLKQAGDVEENPG.

Protein/Peptide 85B-C5: (Met+AG85B CD4 EPITOPE+GSG linker+P2A (self-cleavage peptide)+AIP-C5 peptide):

(SEQ ID NO: 2)
MFQDAYNAAGGHNAVFGSGATNFSLLKQAGDVEENPGPAAQAAVVRFQE
AANKQKQELD.

DNA sequences of the CMV promoter, 85B epitope, peptide 2A, and BGH polyadenylation signal. This gene cassette was used for generating HAdv⁸⁵ or BAdv⁸⁵:

(SEQ ID NO: 3)
GACATTGATTATTGACTAGTTATTAATAGTAATCAATTACGGGGTCATT
AGTTCATAGCCCATATATGGAGTTCCGCGTTACATAACTTACGGTAAAT
GGCCCGCCTGGCTGACCGCCCAACGACCCCCGCCCATTGACGTCAATAA
TGACGTATGTTCCCATAGTAACGCCAATAGGGACTTTCCATTGACGTCA
ATGGGTGGACTATTTACGGTAAACTGCCCACTTGGCAGTACATCAAGTG
TATCATATGCCAAGTACGCCCCCTATTGACGTCAATGACGGTAAATGGC
CCGCCTGGCATTATGCCCAGTACATGACCTTATGGGACTTTCCTACTTG
GCAGTACATCTACGTATTAGTCATCGCTATTACCATGGTGATGCGGTTT
TGGCAGTACATCAATGGGCGTGGATAGCGGTTTGACTCACGGGGATTTC
CAAGTCTCCACCCCATTGACGTCAATGGGAGTTTGTTTTGGCACCAAAA
TCAACGGGACTTTCCAAAATGTCGTAACAACTCCGCCCCATTGACGCAA
ATGGGCGGTAGGCGTGTACGGTGGGAGGTCTATATAAGCAGAGCTCTCT
GGCTAACTAGAGAACCCACTGCTTACTGGCTTATCGAAATTAATACGAC
TCACTATAGGGAGACCCAAGCTTGGTACCGAGCTCGGATCCGCCACCAT
GTTCCAGGACGCCTACAACGCTGCTGGAGGACACAACGCCGTGTTCGGC
AGCGGAGCTACCAACTTTTCCCTGCTGAAGCAGGCTGGCGATGTGGAGG
AGAACCCCGGATAAGCGGCCGCTCGAGCATGCATCTAGAGGGCCCTATT
CTATAGTGTCACCTAAATGCTAGAGCTCGCTGATCAGCCTCGACTGTGC
CTTCTAGTTGCCAGCCATCTGTTGTTTGCCCCTCCCCCGTGCCTTCCTT
GACCCTGGAAGGTGCCACTCCCACTGTCCTTTCCTAATAAAATGAGGAA
ATTGCATCGCATTGTCTGAGTAGGTGTCATTCTATTCTGGGGGGTGGGG
TGGGGCAGGACAGCAAGGGGGAGGATTGGGAAGACAATAGCAGGCATGC
TGGGGATGCGGTGGGCTCTATGG

DNA sequences of the CMV promoter, 85B epitope, peptide 2A, AIP-C5 peptide, and BGH polyadenylation signal. This gene cassette was used for generating HAdv^{85C5} or BAdv^{85C5}:

(SEQ ID NO: 4)
GACATTGATTATTGACTAGTTATTAATAGTAATCAATTACGGGGTCATT
AGTTCATAGCCCATATATGGAGTTCCGCGTTACATAACTTACGGTAAAT
GGCCCGCCTGGCTGACCGCCCAACGACCCCCGCCCATTGACGTCAATAA
TGACGTATGTTCCCATAGTAACGCCAATAGGGACTTTCCATTGACGTCA
ATGGGTGGACTATTTACGGTAAACTGCCCACTTGGCAGTACATCAAGTG
TATCATATGCCAAGTACGCCCCCTATTGACGTCAATGACGGTAAATGGC
CCGCCTGGCATTATGCCCAGTACATGACCTTATGGGACTTTCCTACTTG
GCAGTACATCTACGTATTAGTCATCGCTATTACCATGGTGATGCGGTTT
TGGCAGTACATCAATGGGCGTGGATAGCGGTTTGACTCACGGGGATTTC
CAAGTCTCCACCCCATTGACGTCAATGGGAGTTTGTTTTGGCACCAAAA
TCAACGGGACTTTCCAAAATGTCGTAACAACTCCGCCCCATTGACGCAA
ATGGGCGGTAGGCGTGTACGGTGGGAGGTCTATATAAGCAGAGCTCTCT
GGCTAACTAGAGAACCCACTGCTTACTGGCTTATCGAAATTAATACGAC
TCACTATAGGGAGACCCAAGCTTGGTACCGAGCTCGATATCGGATCCGC
CACCATGTTCCAGGACGCCTACAACGCTGCTGGAGGACACAACGCCGTG
TTCGGCAGCGGAGCTACCAACTTTTCCCTGCTGAAGCAGGCTGGCGATG
TGGAGGAGAACCCAGGACCTGCTGCTCAGGCTGCTGTGGTGAGGTTTCA
GGAGGCCGCTAACAAGCAGAAGCAGGAGCTGGACTAAGCGGCCGCGATA
TCTCGAGCATGCATCTAGAGGGCCCTATTCTATAGTGTCACCTAAATGC
TAGAGCTCGCTGATCAGCCTCGACTGTGCCTTCTAGTTGCCAGCCATCT
GTTGTTTGCCCCTCCCCCGTGCCTTCCTTGACCCTGGAAGGTGCCACTC
CCACTGTCCTTTCCTAATAAAATGAGGAAATTGCATCGCATTGTCTGAG
TAGGTGTCATTCTATTCTGGGGGGTGGGGTGGGGCAGGACAGCAAGGGG
GAGGATTGGGAAGACAATAGCAGGCATGCTGGGGATGCGGTGGGCTCTA
TGG

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

Unless defined otherwise, all technical and scientific terms used herein have the same meaning as commonly understood to one of ordinary skill in the art. As defined herein, the following terms and phrases shall have the meanings set forth below.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), intranasal (in), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular, intranasal and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved. the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

In some illustrative embodiments, this disclosure relates to a recombinant adenovirus vector comprising a heterologous DNA segment encoding mycobacterial Ag85B-p25 epitope (SEQ ID NO: 1), mycobacterial Ag85B-p25 epitope fusion of autophagy-inducing peptide-C5 (SEQ ID NO: 2), or a substantially homologous functional fragment thereof.

In some illustrative embodiments, this disclosure relates to a recombinant adenovirus vector as disclosed herein. wherein said vector comprises a heterologous DNA segment having a SEQ ID NO: 3, SEQ ID NO: 4, or a substantially homologous functional fragment thereof.

In some illustrative embodiments, this disclosure relates to a recombinant adenovirus vector as disclosed herein, wherein said vector is a replication-defective human adenovirus vector, a bovine adenovirus vector or any other adenovirus vector.

In some illustrative embodiments, this disclosure relates to a recombinant adenovirus vector as disclosed herein, wherein said vector is useful as an effective vaccine for protection from infections by a microorganism.

In some illustrative embodiments, this disclosure relates to a recombinant adenovirus vector as disclosed herein, wherein the microorganism is a fungus, a virus, or a bacteria.

In some illustrative embodiments, this disclosure relates to a recombinant adenovirus vector as disclosed herein, wherein the microorganism is Mycobacterium tuberculosis (Mtb).

In some illustrative embodiments, this disclosure relates to a recombinant adenovirus vector as disclosed herein, wherein said vector is useful as an effective vaccine delivered nasally.

In some illustrative embodiments, this disclosure relates to a recombinant adenovirus vector as disclosed herein, wherein said vector is an effective vaccine for tuberculosis.

In some illustrative embodiments, this disclosure relates to a recombinant adenovirus vector as disclosed herein, wherein said vector is useful as an effective vaccine by way of infecting dendritic cells (DCs) that expressed a unique transcriptome of genes regulating antigen processing.

In some illustrative embodiments, this disclosure relates to a recombinant adenovirus vector as disclosed herein, wherein the heterologous DNA segment is operationally fused in an expression cassette with an autophagy-inducing peptide such as AIP-C5.

In some illustrative embodiments, this disclosure relates to a method of therapeutically or prophylactically immunizing a subject comprising administering to the subject a recombinant adenovirus vector having a heterologous DNA segment encoding mycobacterial Ag85B-p25 epitope (SEQ ID NO: 1), mycobacterial Ag85B-p25 epitope fusion of autophagy-inducing peptide-C5 (SEQ ID NO: 2), or a substantially homologous functional fragment thereof.

In some illustrative embodiments, this disclosure relates to a method of therapeutically or prophylactically immunizing a subject as disclosed herein, wherein said vector comprises a heterologous DNA segment having a SEQ ID NO: 3, SEQ ID NO: 4, or a substantially homologous functional fragment thereof.

In some illustrative embodiments, this disclosure relates to a method of therapeutically or prophylactically immunizing a subject, wherein said vector is a replication defective human adenovirus vector, a bovine adenovirus vector or any other adenovirus vector.

In some illustrative embodiments, this disclosure relates to a method of therapeutically or prophylactically immunizing a subject, wherein said vector is useful as an effective vaccine for protection from infections by a microorganism.

In some illustrative embodiments, this disclosure relates to a method of therapeutically or prophylactically immunizing a subject, wherein the microorganism is a fungus, a virus, or a bacteria.

In some illustrative embodiments, this disclosure relates to a method of therapeutically or prophylactically immunizing a subject, wherein the microorganism is Mycobacterium tuberculosis (Mtb).

In some illustrative embodiments, this disclosure relates to a method of therapeutically or prophylactically immunizing a subject, wherein said vector is useful as an effective mucosal vaccine.

In some illustrative embodiments, this disclosure relates to a method of therapeutically or prophylactically immunizing a subject, wherein said vector is useful as an effective vaccine delivered nasally.

In some illustrative embodiments, this disclosure relates to a method of therapeutically or prophylactically immunizing a subject, wherein said vector is an effective vaccine for tuberculosis.

In some illustrative embodiments, this disclosure relates to a method of therapeutically or prophylactically immunizing a subject, wherein said vector is useful as an effective vaccine by way of infecting dendritic cells (DCs) that expressed a unique transcriptome of genes regulating antigen processing.

In some illustrative embodiments, this disclosure relates to a method of therapeutically or prophylactically immunizing a subject, wherein the heterologous DNA segment is operationally fused in an expression cassette with an autophagy-inducing peptide such as AIP-C5.

In some illustrative embodiments, this disclosure relates to a method of therapeutically or prophylactically immunizing a subject, wherein the subject is a human being or an animal.

Yet in some illustrative embodiments, this disclosure relates to a pharmaceutical composition for therapeutically or prophylactically immunizing a subject comprising a recombinant adenovirus vector having a heterologous DNA segment encoding mycobacterial Ag85B-p25 epitope (SEQ ID NO: 1), mycobacterial Ag85B-p25 epitope fusion of autophagy-inducing peptide-C5 (SEQ ID NO: 2), or a substantially homologous functional fragment thereof, together with one or more pharmaceutically acceptable carriers, diluents, or excipients.

In some illustrative embodiments, this disclosure relates to a pharmaceutical composition for therapeutically or prophylactically immunizing a subject as disclosed herein, wherein said vector comprises a heterologous DNA segment having a SEQ ID NO: 3, SEQ ID NO: 4, or a substantially homologous functional fragment thereof.

In some illustrative embodiments, this disclosure relates to a pharmaceutical composition for therapeutically or prophylactically immunizing a subject as disclosed herein, wherein said vector is a replication-defective human adenovirus vector, a bovine adenovirus vector or any other adenovirus vector.

In some illustrative embodiments, this disclosure relates to a pharmaceutical composition for therapeutically or prophylactically immunizing a subject as disclosed herein, wherein said vector is useful as an effective vaccine for protection from infections by a microorganism.

In some illustrative embodiments, this disclosure relates to a pharmaceutical composition for therapeutically or prophylactically immunizing a subject as disclosed herein, wherein the microorganism is a fungus, a virus, or a bacteria.

In some illustrative embodiments, this disclosure relates to a pharmaceutical composition for therapeutically or prophylactically immunizing a subject as disclosed herein, wherein the microorganism is Mycobacterium tuberculosis (Mtb).

In some illustrative embodiments, this disclosure relates to a pharmaceutical composition for therapeutically or prophylactically immunizing a subject as disclosed herein, wherein said vector is useful as an effective mucosal vaccine.

In some illustrative embodiments, this disclosure relates to a pharmaceutical composition for therapeutically or prophylactically immunizing a subject as disclosed herein, wherein said vector is useful as an effective vaccine delivered nasally.

In some illustrative embodiments, this disclosure relates to a pharmaceutical composition for therapeutically or prophylactically immunizing a subject as disclosed herein, wherein said vector is an effective vaccine for tuberculosis.

In some illustrative embodiments, this disclosure relates to a pharmaceutical composition for therapeutically or prophylactically immunizing a subject as disclosed herein, wherein said vector is useful as an effective vaccine by way of infecting dendritic cells (DCs) that expressed a unique transcriptome of genes regulating antigen processing.

In some illustrative embodiments, this disclosure relates to a pharmaceutical composition for therapeutically or prophylactically immunizing a subject as disclosed herein, wherein the heterologous DNA segment is operationally fused in an expression cassette with an autophagy-inducing peptide such as AIP-C5.

In some illustrative embodiments, this disclosure relates to a pharmaceutical composition for therapeutically or prophylactically immunizing a subject as disclosed herein, wherein the subject is a human being or an animal.

The following non-limiting exemplary embodiments are included herein to further illustrate the invention. These exemplary embodiments are not intended and should not be interpreted to limit the scope of the invention in any way. It is also to be understood that numerous variations of these exemplary embodiments are contemplated herein.

Tuberculosis kills 1.5 million people including children each year and the BCG vaccine offers partial protection from lung disease. We developed mucosal vaccines expressing the autophagy-inducing peptide-C5 (AIP-C5) and mycobacterial Ag85B-p25 epitope using replication-defective human adenovirus (HAdv^{85C5}) and bovine adenovirus (BAdv^{85C5}) vectors. BAdv^{85C5}-infected dendritic cells (DCs) expressed a unique transcriptome of genes regulating antigen processing compared to HAdv^{85C5}-infected DCs. BAdv^{85C5}-infected DCs showed an enhanced Galectin-3/8 and autophagy-dependent in vitro Ag85B-p25 epitope presentation to CD4 T cells. Naïve or BCG-vaccinated mice were intranasally vaccinated using HAdv^{85C5} or BAdv^{85C5} followed by aerosolized Mycobacterium tuberculosis (Mtb). BAdv^{85C5} protected mice against tuberculosis both as a booster after BCG vaccine (>1.4-log 10 reduction in Mtb lung burden) and as a single intranasal dose (>0.5-log 10 reduction). Protection was associated with a robust expansion of CD4 and CD8 effector (T_{EM}), central memory (T_{CM}), and CD103⁺/CD69⁺ lung resident memory (T_{RM}) T cells. Thus, BAdv^{85C5} is a novel mucosal vaccine for tuberculosis.

The viral vector-based vaccine platforms for Mtb are excellent in inducing both systemic and mucosal immunity in mice. However, respiratory mucosal booster vaccines following BCG immunization may still fall short from protecting neonates from tuberculosis. This is likely due to a defective expansion of T_{RM} during influenza infection in neonatal mice and human neonates (Zens et al., 2017). Related studies show that the neonatal immune system is functional but physiologically immature, requiring vaccine adjuvants to elicit a robust T-helper immunity regulating cytokines like IL-12, TNF-α and IL1-β in APCs (Saso and Kampmann, 2017; Yu et al., 2018). The relevance of an immature neonatal immune system is also underscored by the requirement of boosters for childhood vaccines like DPT and MMR. Therefore, the intrinsic defects of BCG vaccine in APCs combined with an immature neonatal immune system may explain the high prevalence of lung tuberculosis and dissemination of Mtb into the brain in neonates despite BCG vaccination.

To design a novel vaccine platform capable of antigen presentation through autophagy, we developed a bovine adenovirus (BAdv)-based tuberculosis vaccine expressing the immunodominant mycobacterial Ag85B-p25 epitope along with AIP-C5. Immunogenicity of BAdv vector-based vaccines do not interfere with the pre-existing adenovirus antibodies (Singh et al., 2008; Bangari and Mittal, 2006; Sharma et al., 2010a). We demonstrate herein that our engineered mucosal vaccine augments the ability of APCs to process and present the Ag85B-p25 epitope to CD4 T cells. Our novel nasal vaccine protected mice following aerosolized challenge with Mtb, both as a booster after the BCG vaccine or on its own. There was a robust expansion of CD4 and CD8 effector (T_{EM}), central memory (T_{CM}), and CD103⁺/CD69⁺ resident memory (T_{RM}) T cells in the lungs of vaccinated mice.

### Results

### Generation of BAdv- or HAdv-Based Vectors Expressing the Mtb Ag85B-p25 Epitope With or Without AIP-C5

Adenovirus (Adv) vector-based vaccines elicit both humoral and cell-mediated immune (CMI) responses (Bangari and Mittal, 2006; Vemula and Mittal, 2010) due to the adjuvant- like effect of Adv vectors by activating the innate immune system through both Toll-like Receptor (TLR)-dependent and TLR-independent pathways (Sharma et al., 2010a; Zhu et al., 2007). Influenza is one of the significant respiratory diseases in humans, animals, and birds. Adv vector-based influenza vaccines have conferred protective efficacy in both animal models (Cao et al., 2016; Hoelscher et al., 2006; Hoelscher et al., 2007) and clinical trials in humans (Barouch et al., 2018; Ledgerwood et al., 2017; Smaill et al., 2013; Van Kampen et al., 2005; van Zyl-Smit et al., 2017).

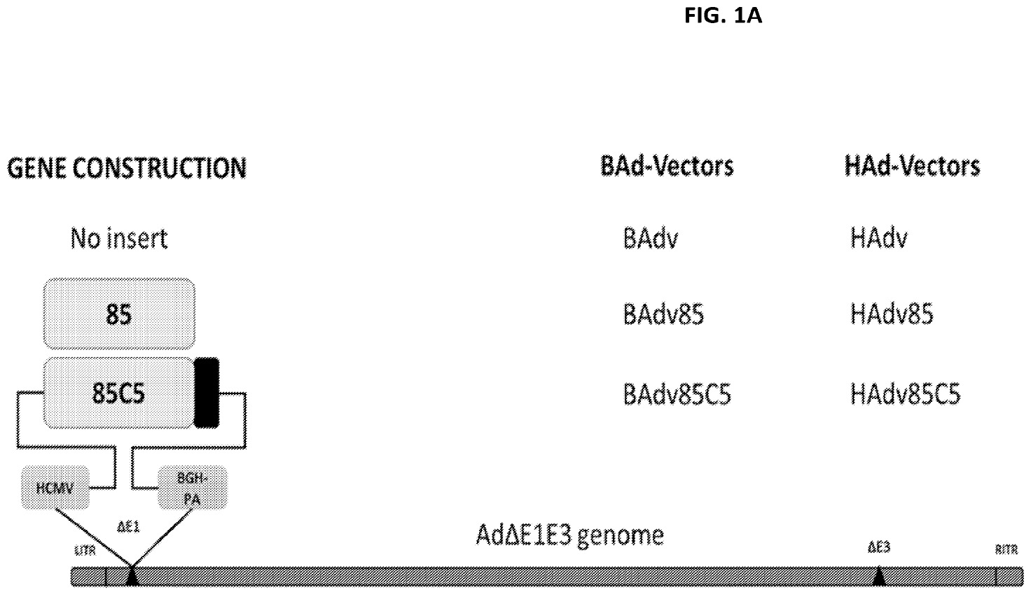

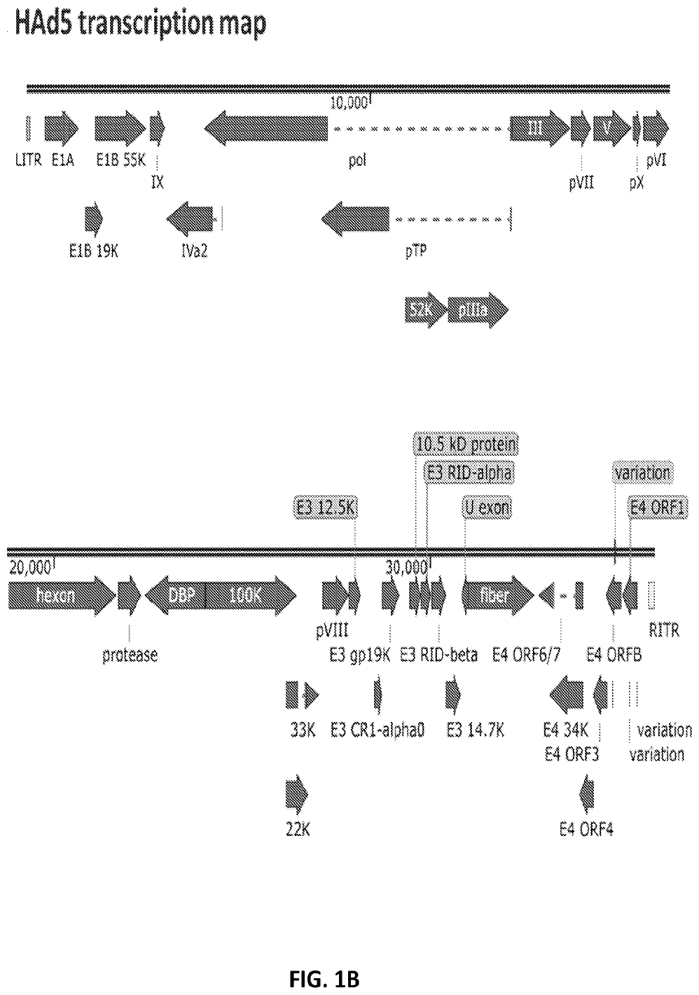

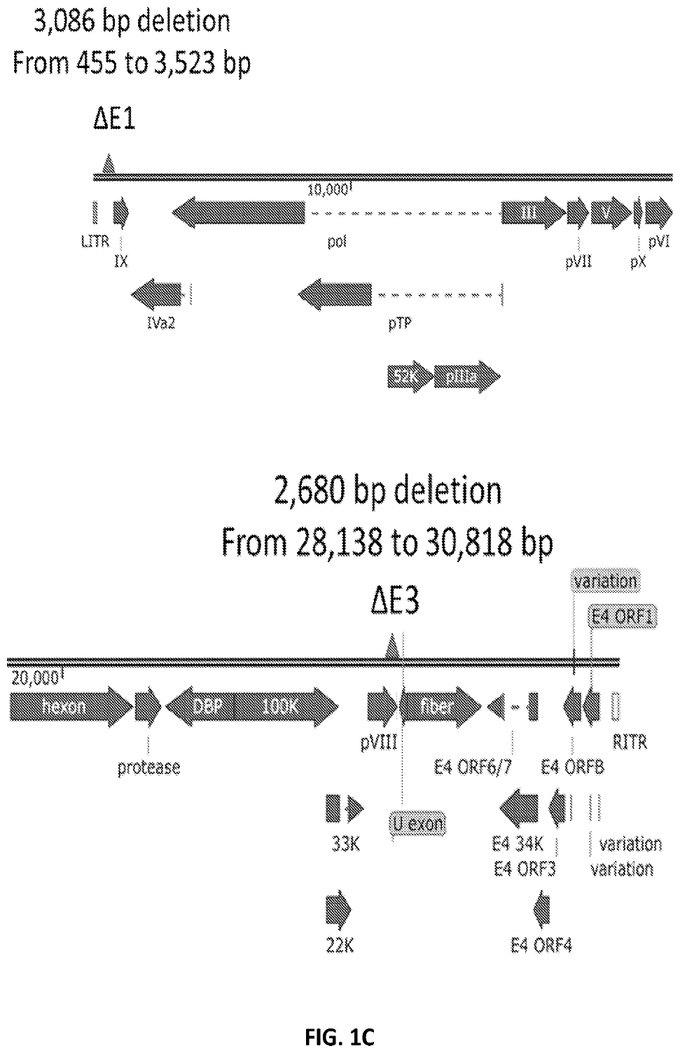

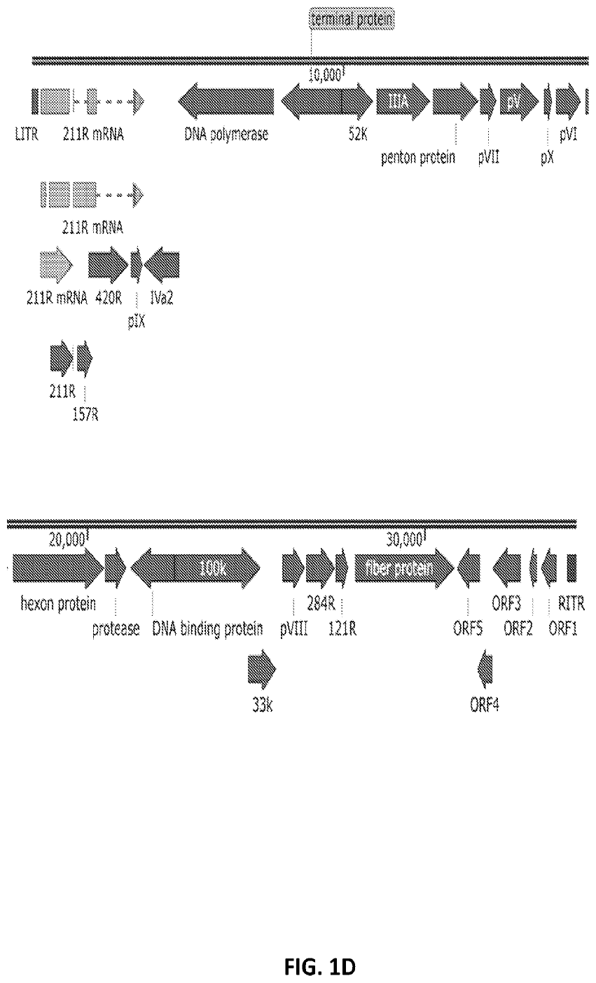

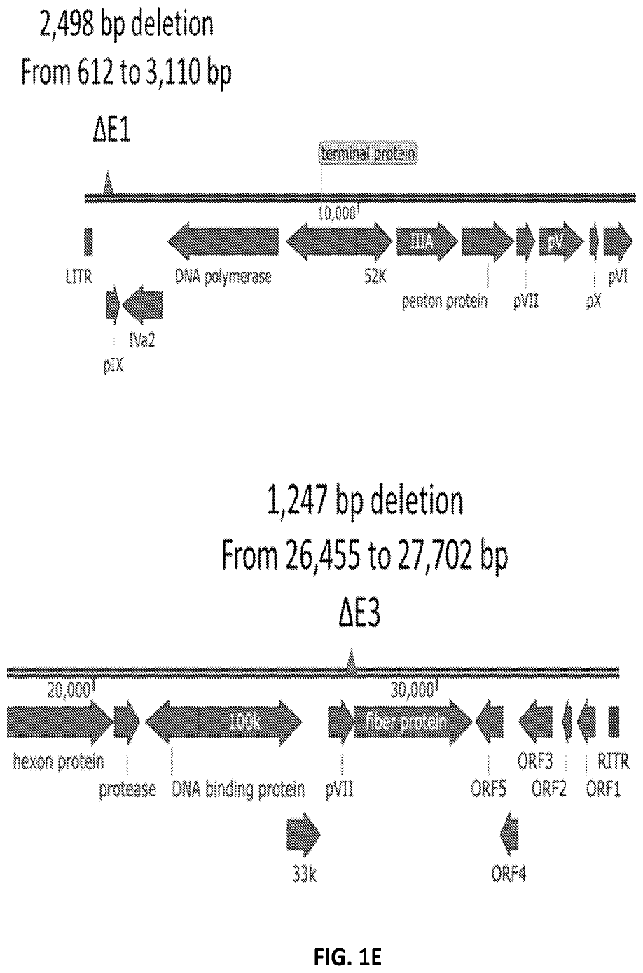

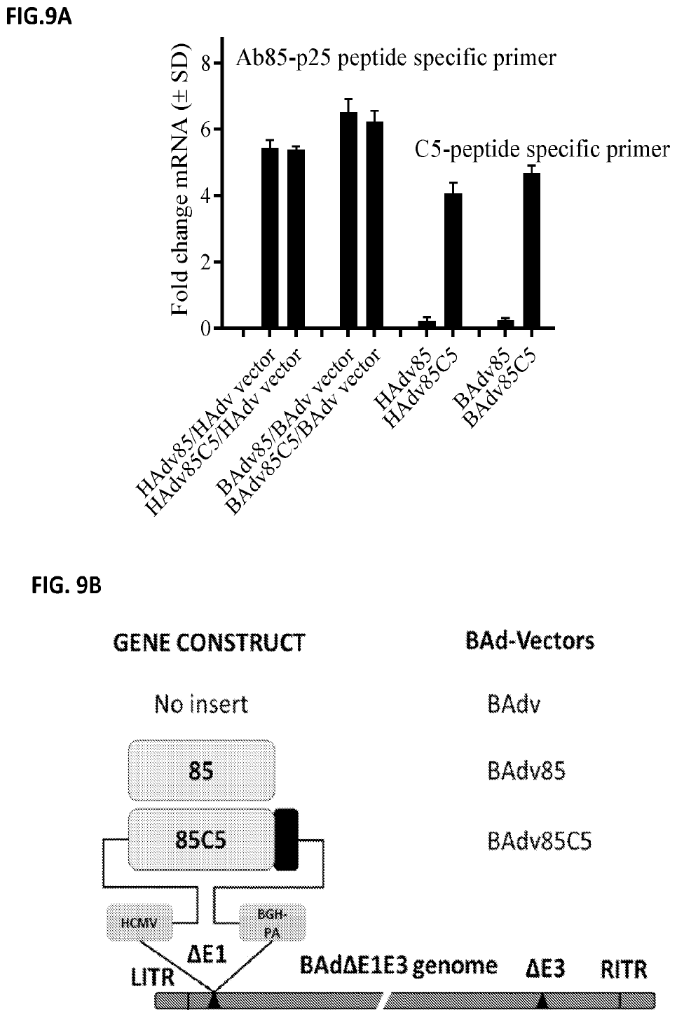

We expressed the Mtb Ag85B-p25 epitope with or without AIP-C5 in the replication-defective BAdv or HAdv vector system. The Mtb Ag85B-p25 epitope (85) or 85+AIP-C5 (85C5) gene cassette was under the control of the immediate early cytomegalovirus (CMV) promoter and bovine growth hormone (BGH) polyadenylation signal. The generated vectors BAdv⁸⁵ (expressing 85), BAdv^{85C5} (expressing 85C5), HAdv⁸⁵ (expressing 85), or HAdv^{85C5} (expressing 85C5) (FIG. 1) were characterized for the presence of the gene cassette and its expression by sequencing and RT-PCR (FIGS. 9A-9B). The BAdv vector platform was selected for our Mtb vaccine because it induced robust humoral and CMI responses in mice compared to the HAdv vector system (Sayedahmed et al., 2018). HAdv vectors were used in several experiments for comparison.

### Replication-Defective BAdv^{85C5} Vaccine Efficiently Infects Immature Mouse DCs and Induces a Unique Transcriptome

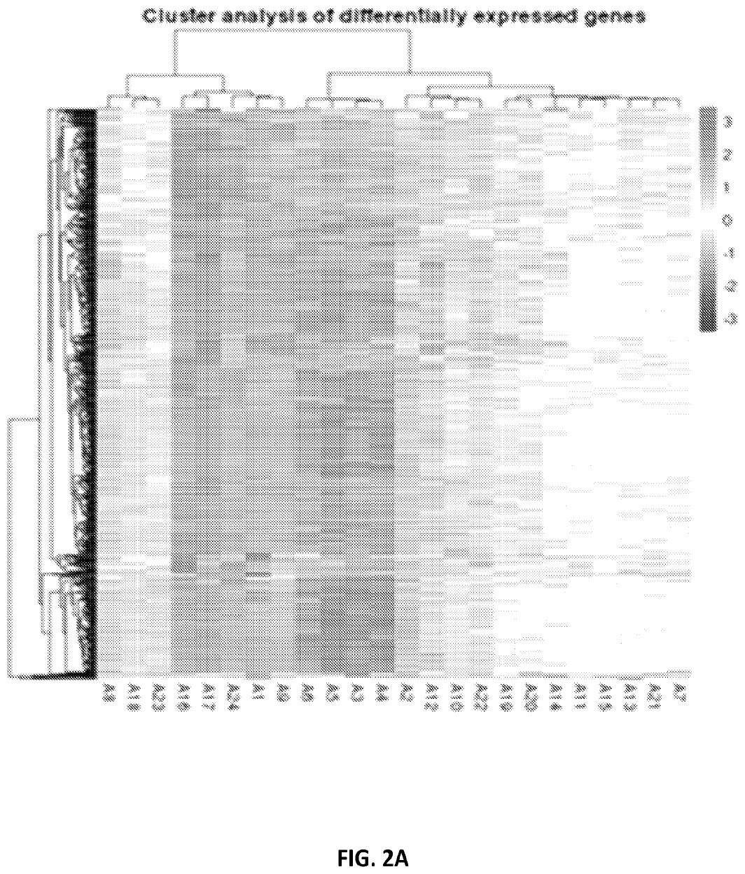

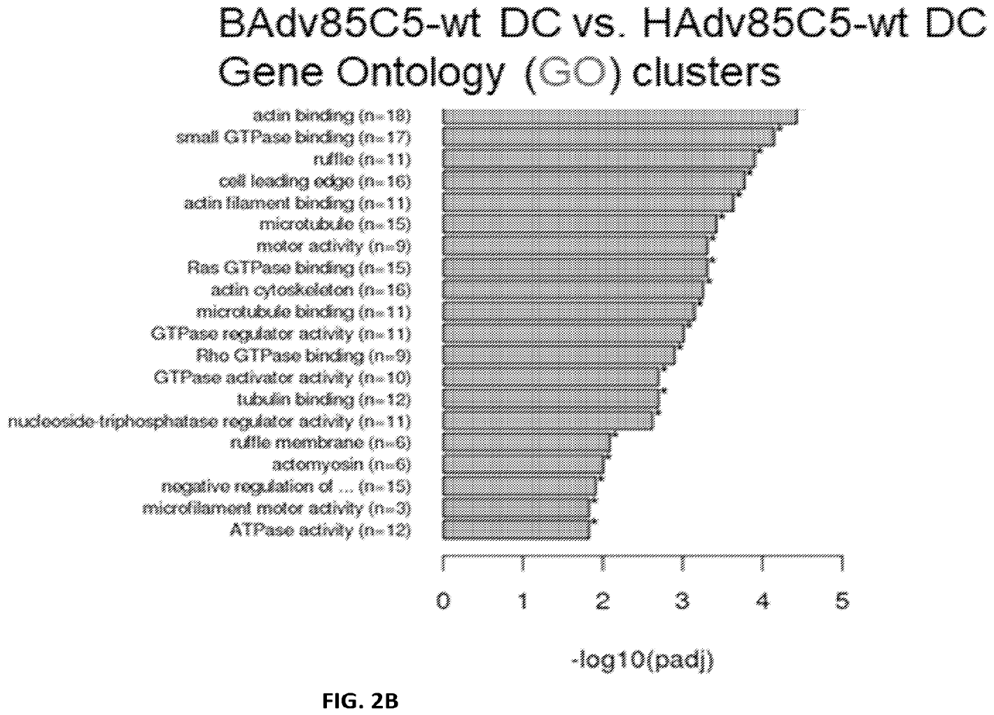

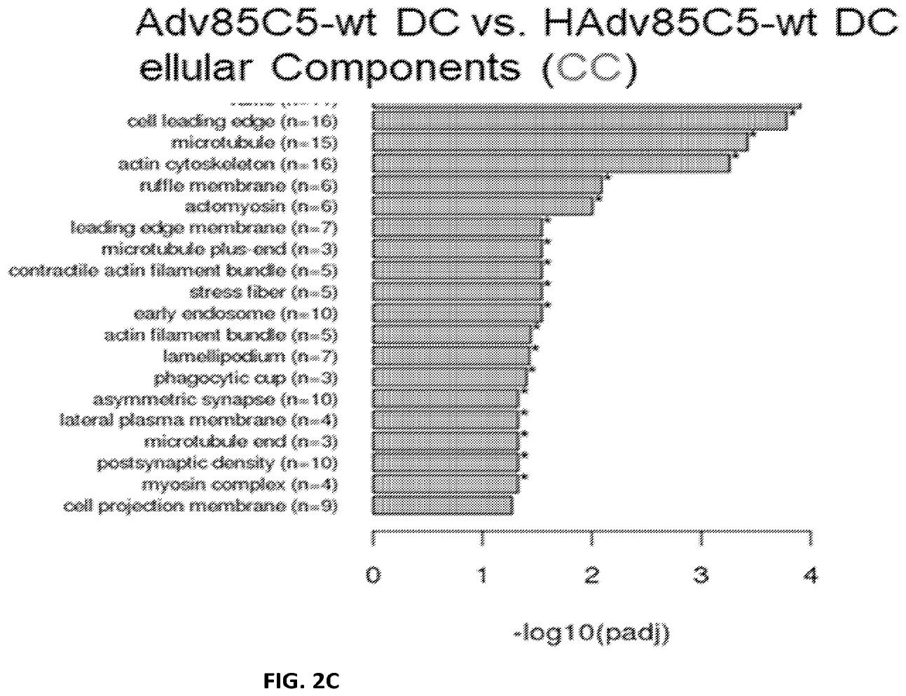

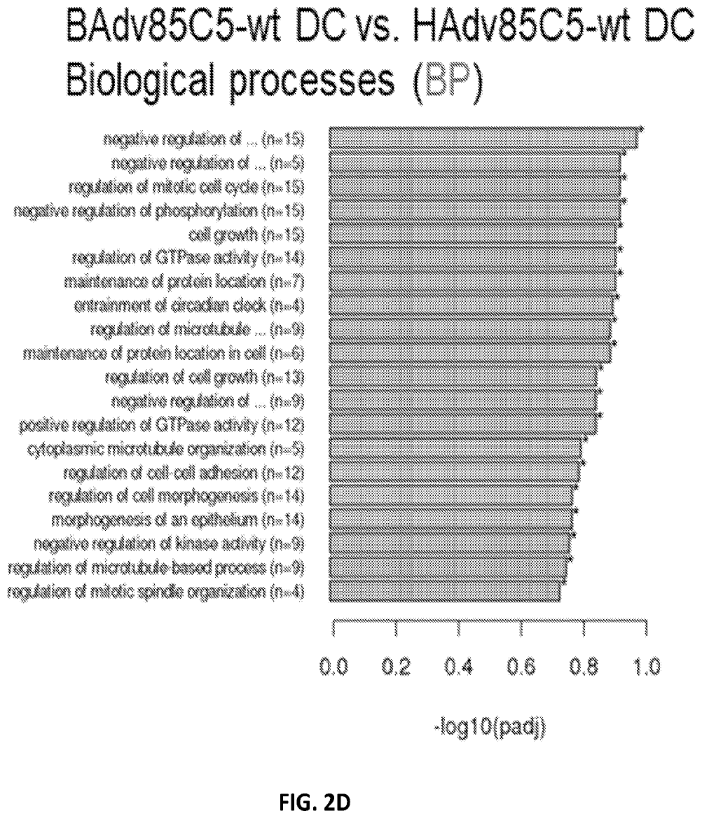

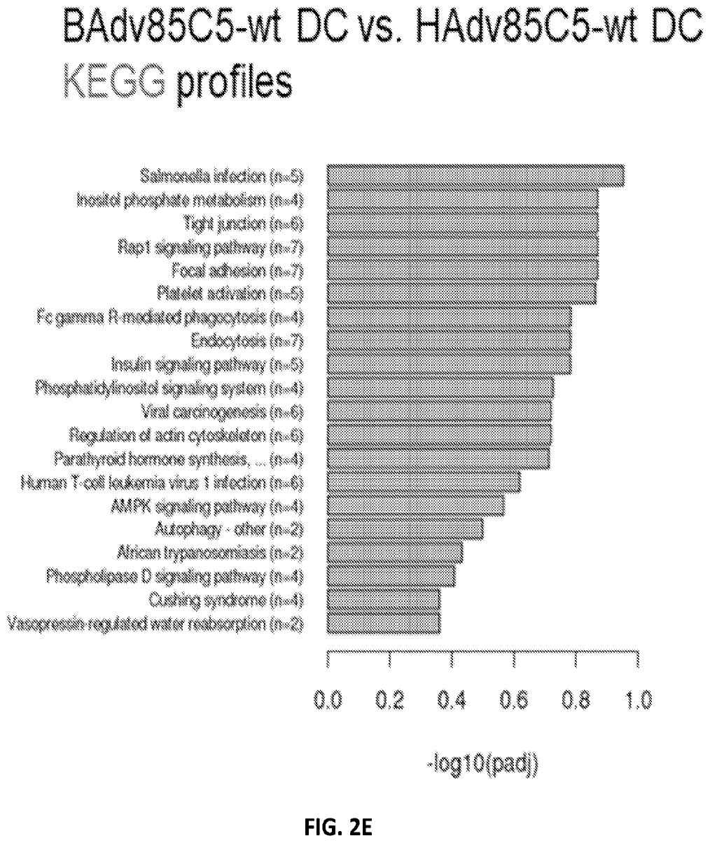

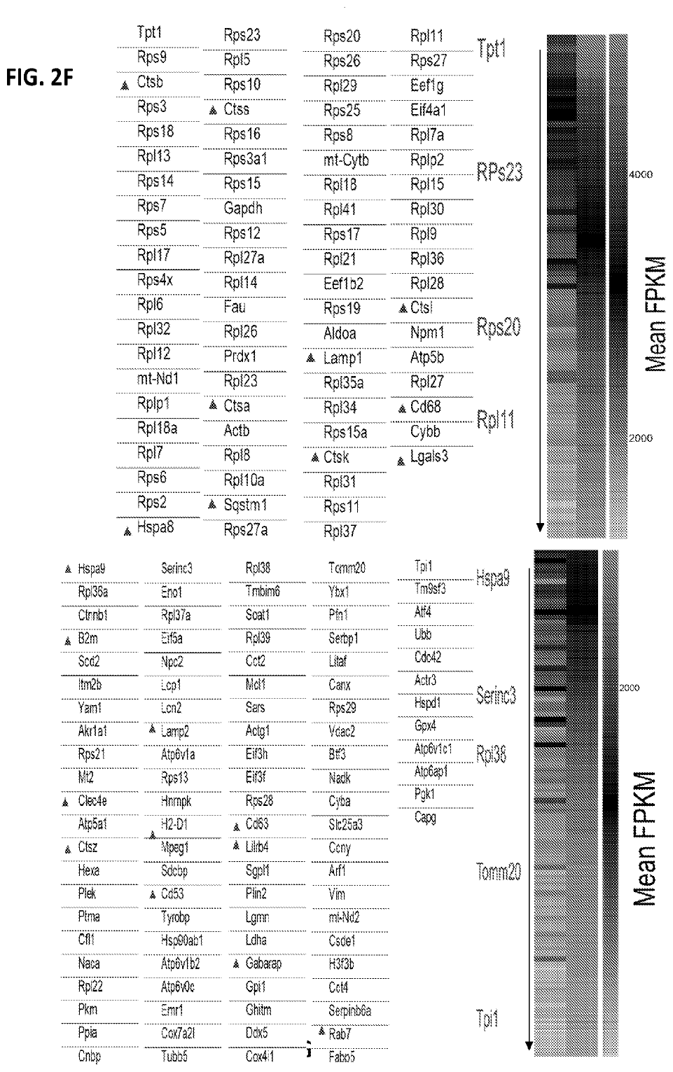

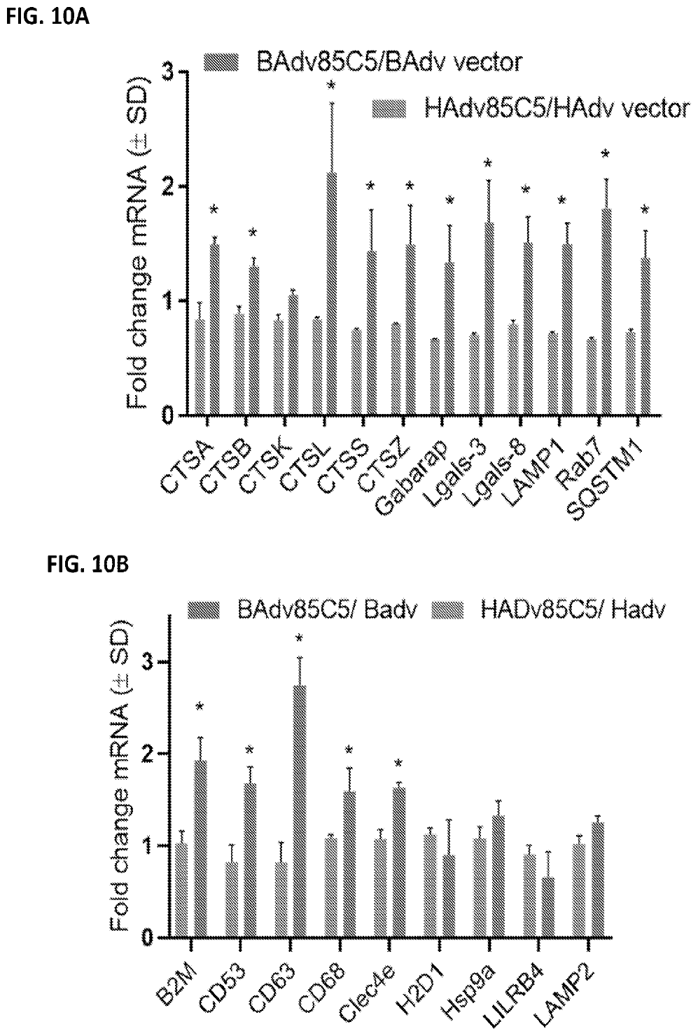

To characterize the virus induced transcriptome, bone marrow-derived CD11c⁺ immature DCs from wt-C57BL/6 mice and autophagy-deficient ATG7KO-DC mice (Chen et al., 2014) were infected with BAdv^{85C5}, BAdv vector, HAdv^{85C5}, or HAdv vector followed by RNAseq analysis (Novogene USA). FIG. 2A illustrates the heat maps of differential gene expressions (DEGs) (n=2) of BAdv- and HAdv-derived vaccine or vector infected wt-DCs and ATG7KO-DCs. Clusterprofiler analysis showed a relative enrichment of multiple genes in BAdv^{85C5}- vs. HAdv^{85C5}-infected wt-DCs. Gene ontology (GO; BP, CC), and KEGG (Kyoto Encyclopedia of Genes and Genomes) profiles are illustrated in FIGS. 2B-2E. Intriguingly, heatmaps of transcripts derived from RNAseq expressed as FPKMs (Fragments per kilo base per million mapped reads; n=2) show that BAdv^{85C5} upregulated the expression of gene clusters involved in antigen processing and endosome sorting to lysosomes. For example, cathepsins (CTSB, CTSA, CTSS, CTSK, and CTSZ) were enriched in BAdv^{85C5} infected DCs compared to HAdv^{85C5} (FIG. 2F). Cathepsins proteolytically cleave antigens into peptides and help their loading onto the MHC-II, which are in turn, exported to the plasma membrane for activation of CD4 T cells. Furthermore, BAdv^{85C5} upregulated the genes of the MHC pathway (H2-D1, B2m), and LAMP1, LAMP2, Hspa8, Hspa9, CD68 (LAMP4), and Galectin3 (Gal3; aka.Lgals-3) which participate during the sorting of antigens and pathogens into the lysosomes (Perez-Montesinos et al., 2017; Jia et al., 2018; Li et al., 2020). Transcripts for Lagls-8 were also found enriched BAdv^{85C5}-infected DCs (mean FPKMs BAdv^{85C5}750±55 vs. HAdv^{85C5}210±15; p<0.001; not shown). Finally, Gabarap (LC3 family) and RAB7 were up-regulated in BAdv^{85C5}-infected DCs and the latter two are key players during autophagolysosome fusion (Munz, 2015; Schaaf et al., 2016). Many of these genes were also selectively upregulated by BAdv^{85C5}-infected wt-DCs compared to ATG7KO-DCs suggesting that, BAdv^{85C5}-upregulated genes are associated with autophagy. qPCR validation of the genes involved during antigen sorting and processing is shown FIG. 10 and discussed in the context of vaccine induced immunogenicity below.

### Replication-Defective BAdv^{85C5} Vaccine is Rapidly Internalized by DCs and Localizes to Autophagolysosomal Compartments

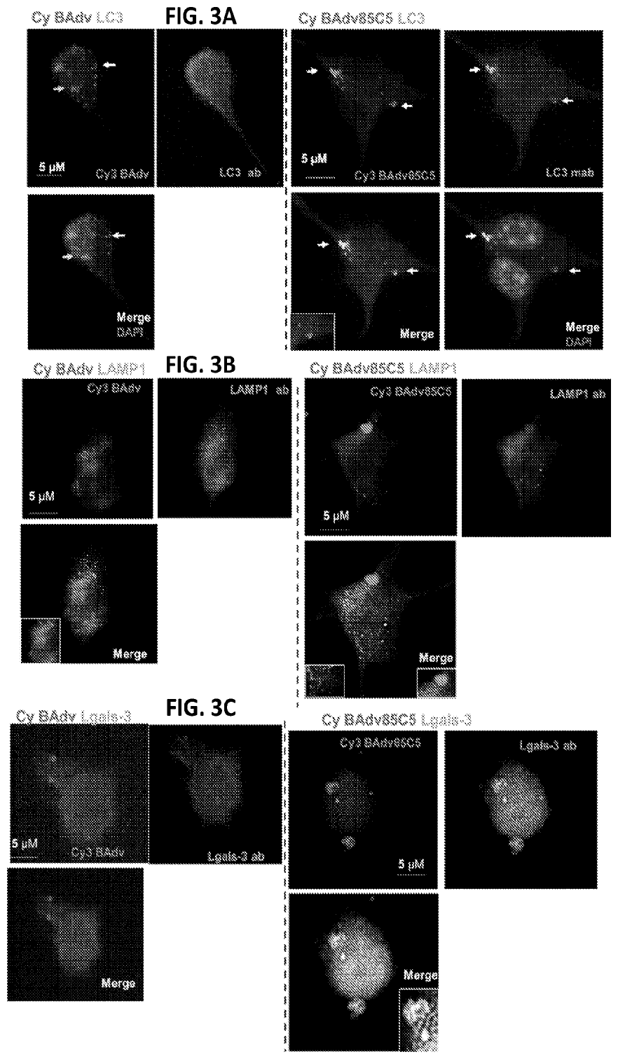

DCs are essential for vaccine-induced immune responses in both mice and humans (Segura and Amigorena, 2015). Earlier studies indicated that Cy3-fluorescent-labeled Adv retain >98% infectivity (Leopold et al., 1998); therefore, Cy3-labeled BAdv^{85C5} or BAdv vector was used for infection of mouse CD11c⁺ immature DCs followed by confocal microscopy. Since Adv can interact with the autophagy pathway and microtubule-associated light chain-3 (LC3) is a known marker of autophagosomes, LC3 was used as a marker for the virus containing endosomes (Hendrickx et al., 2014). Cy3-BAdv^{85C5} and Cy3-BAdv vector were rapidly internalized and FIG. 3A illustrates their uptake; both were found distributed between the cytosol and nucleus after a 4 h infection cycle similar to an earlier report (Bailey et al., 2003). Endosomes colocalizing with the LC3 marker were quantitated which indicate an increased labeling of Cy3-BAdv^{85C5} endosomes compared to those containing Cy3-BAdv vector. Likewise, Lysosome-associated membrane protein-1 (LAMP1) labeling was higher suggesting that Cy3-BAdv^{85C5} localizes more into autophagolysosomes than the vector (FIG. 3B). Earlier studies indicate that autophagy plays a role during the translocation of Adv capsids from the endosome to the nuclear pore complex and during this process, lectin-like intracellular receptors such as Galectin3 and Galctin8 (aka.Lgals-8) are associated with the vesicular transport of Adv (Maier et al., 2012; Montespan et al., 2017). We found that BAdv^{85C5} infected DCs showed a stronger enrichment of Lgals-3 than Lgals-8 (FIG. 3C). It is pertinent to recall here that HAdv infects immature mouse DCs less effectively than a HAdv vector engineered to target DCs (Sharma et al., 2018). In contrast, DCs efficiently internalized both Cy3-BAdv^{85C5} and Cy3-BAdv vector. Therefore, similar to HAdv^{85C5} BAdv^{85C5} is also associated with the autophagy pathways in DCs with an enhanced expression of genes regulating endosome traffic.

### Replication-Defective BAdv^{85C5} Vaccine Enhances Autophagy-Dependent and -Independent Antigen Presentation in Both Mouse DCs and Human MΦs

Previous studies show that HAdv vectors expressing mycobacterial antigens protect mice and macaques against TB variably; although, human studies have not been encouraging (Hoft et al., 2012; Rodo et al., 2019; Darrah et al., 2019). Because BAdv^{85C5} induced robust gene expression in DCs, we sought to further define whether it increases the immunogenicity of DCs in comparison with HAdv^{85C5}.

A major function of vaccine ingested DCs is an efficient antigen processing and activation of CD4 and CD8 T cells through MHC-II and MHC-I pathway, respectively. Whereas, vaccines degraded in lysosomes are routed to MHC-II pathway, proteasome digested peptides of vaccines are routed through MHC-I. We demonstrated earlier that autophagy can increase MHC-II-dependent presentation of mycobacterial Ag85B (Jagannath et al., 2009), whereas, autophagy was also found to increase MHC-I-dependent presentation of antigens in APCs (Lee et al., 2010). To decipher the antigen processing mechanisms of DCs, we developed an ex vivo assay initially developed Cliff Harding's group, where, BCG- or Mtb-infected APCs rapidly present an Ag85B-derived p25 epitope to BB7 CD4 T cells in vitro (Ramachandra et al., 1999, 2001) This assay has been extensively used to measure the immunogenicity of BCG and Mtb mutants by us and others (Singh et al., 2006; Soualhine et al., 2007; Saini et al., 2016). We reported earlier that BCG85B (BCG overexpressing full-length Ag85B) or BCG85BC5 (BCG overexpressing full-length Ag85B and AIP-C5) induce robust autophagy, increasing p25 presentation and elicit IL-2 from BB7 CD4 T cells (Jagannath et al., 2009; Khan et al., 2019). Importantly, in vitro antigen presentation responses correlated directly with their efficacy against tuberculosis in mice (Jagannath et al., 2009; Khan et al., 2019). Since the BAdv^{85C5} vaccine expressed Ag85Bd-p25 epitope and AIP-C5, we first sought to determine the antigen processing mechanisms in the presence or absence of autophagy.

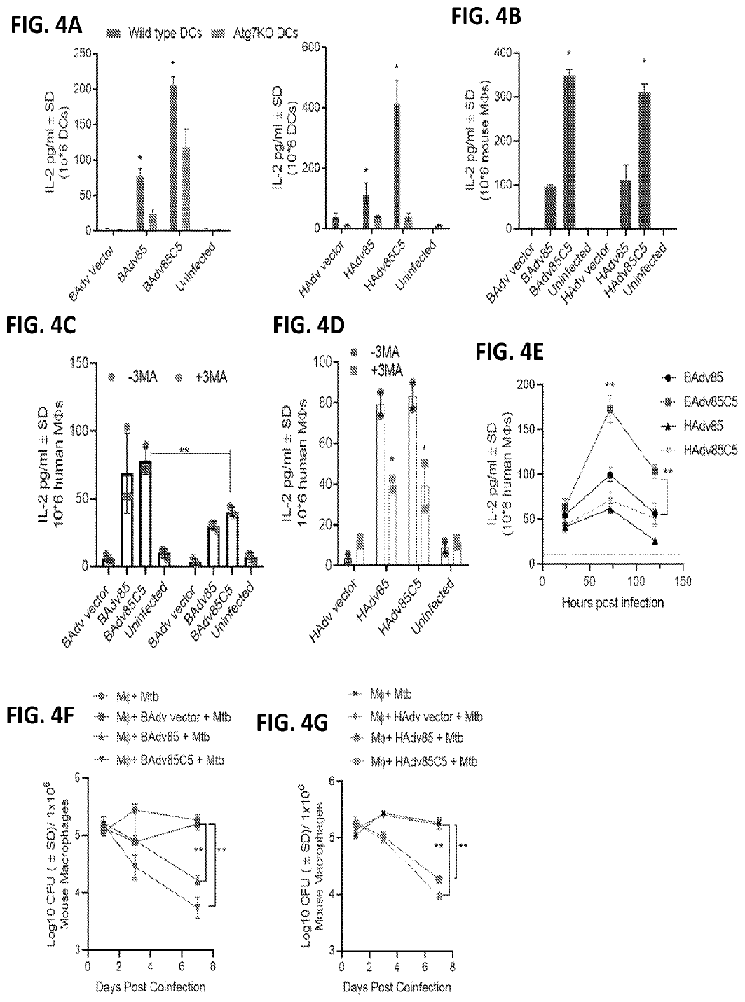

Autophagy begins with an intracellular membrane vesicle nucleation, vesicle elongation and autophagophore formation, which encloses pathogens into an autophagosome. The latter fuse with the lysosomes which in turn, degrades pathogens for the production of antigenic peptides through Cathepsin proteases. This process, also known as macroautophagy, involves several autophagy-regulating genes (ATGs) of which, ATG7 and ATG5 are key genes, although alternative pathways exist (Nishida et al., 2009). To determine that autophagy plays a role during a virus vector-based vaccine-induced antigen presentation, wt-DCs and ATG7KO-DCs were infected with BAdv^{85C5} or HAdv^{85C5} followed by antigen presentation. ATG7 deficiency in DCs led to a significant reduction of antigen presentation after infection with either BAdv^{85C5} or HAdv^{85C5} (FIG. 4A). Supporting these data, wt-DCs and ATG7KO-DCs infected with BAdv^{85C5} showed striking differences in the gene expression, strengthening the contention that autophagy is important during the antigen processing of BAdv^{85C5} vaccine. BAdv^{85C5} also induced an upregulation of antigen presentation in MΦs comparable to HAdv^{85C5} (FIG. 4B) and induced better gene expression compared to HAdv^{85C5} vaccine.

Since the BAdv^{85C5} vaccine platform is being developed for human neonates following BCG vaccination, human CD14⁺ MΦs were untreated or treated with 3-methyladenine (50 μM) to inhibit autophagy followed by infections with BAdv^{85C5} or HAdv^{85C5}. Even in human MΦs, the blockade of autophagy reduced the antigen presentation by BAdv^{85C5} or HAdv^{85C5} (FIG. 4C-D). In addition, BAdv^{85C5} induced an elevated and sustained antigen presentation compared to HAdv^{85C5} in human MΦs (FIG. 4E). It is known that autophagy-mediated delivery of mycobacteria leads to their degradation in lysosomes reducing their viability (Jagannath et al., 2009). Thus, mouse CD14⁺ MΦs were infected with vectors or vaccines followed by infection with Mtb. FIG. 4F-G demonstrate that both BAdv⁸⁵ and BAdv^{85C5} reduce the viability of intracellular Mtb. Together, these data indicate that BAdv^{85C5} induces a robust autophagy-mediated antigen presentation to CD4 T cells in mouse DCs, mouse MΦs, and human MΦs.

### Replication-Defective BAdv^{85C5} Vaccine Enhances Antigen Presentation Through Galectins and Cathepsin Expression in DCs

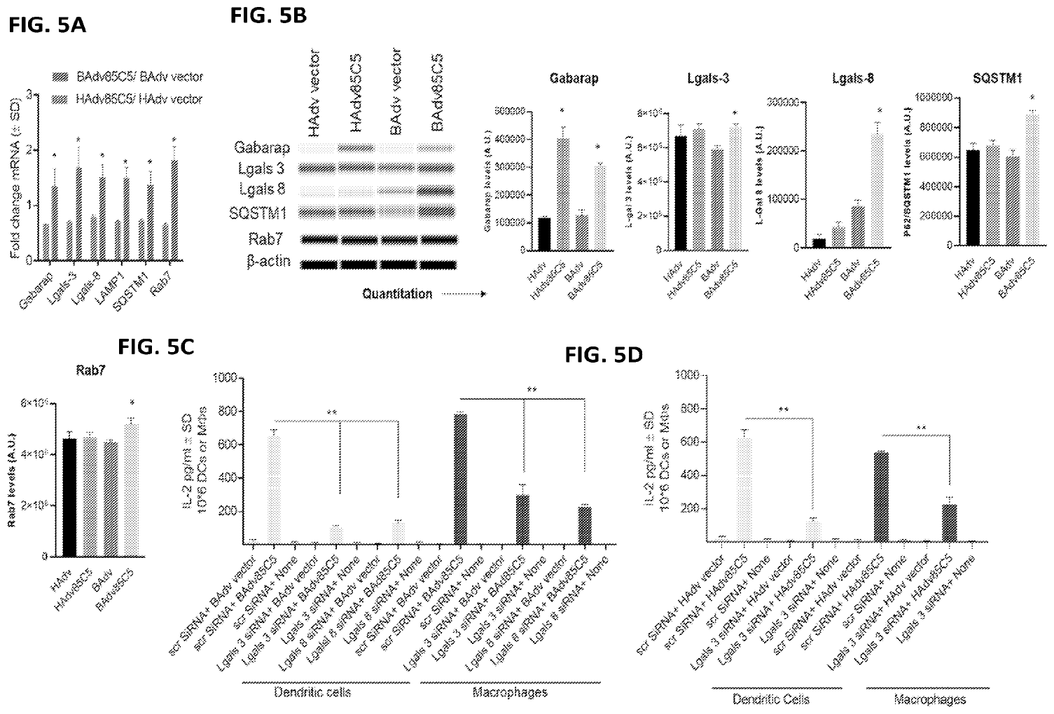

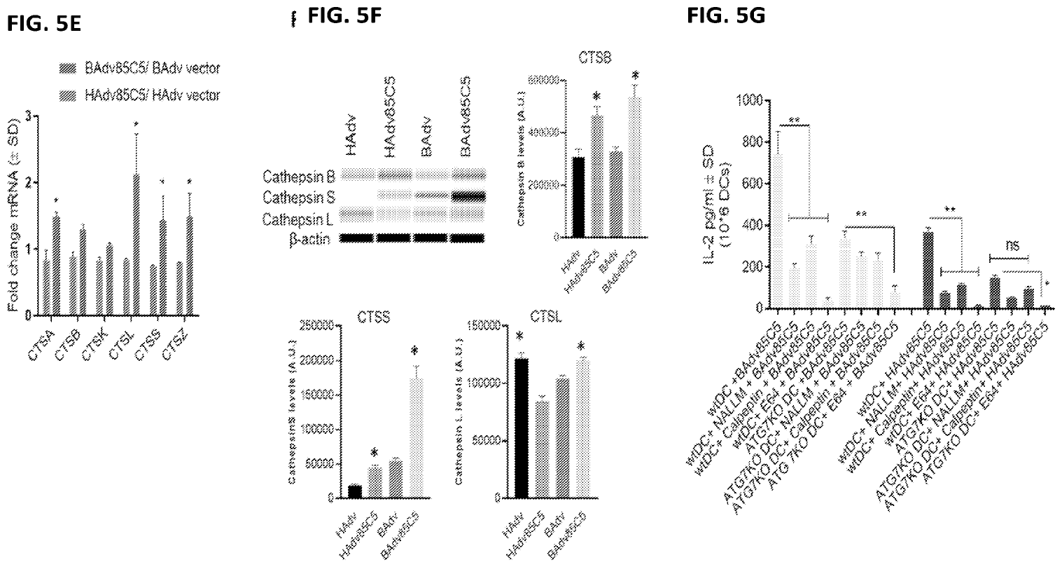

Many studies indicate that autophagy plays a role during the translocation of Adv capsids from the endosome to the nuclear pore complex (Hendrickx et al., 2014). During this process, lectin-like intracellular receptors like Galectins, specifically, Lgals-/(Gal3) and Lgals-3 (Gal8) are associated with the vesicular transport of Adv (Hendrickx et al., 2014); although, HAdv5 infection of A549 epithelial cells strongly down-regulated Gal1 and Gal3 expression (Trinh et al., 2013). Transcriptomic studies show that Lgals-3 (FIG. 2) and Lgals-8 transcripts were upregulated in BAdv^{85C5} infected DCs. qPCR validation showed that BAdv^{85C5} induced mRNA transcripts for Lgals-3 and Lgals-8 better than HAdv85C5 infected DCs (FIG. 5A). Interestingly, western blots indicated that Lgals-3 protein was uniformly induced in DCs by both vectors and vaccines whereas, Lgals-8 was induced strongly in BAdv^{85C5}-infected DCs (FIG. 5B).

qPCR and western blot analysis of BAdv^{85C5} and HAdv^{85C5} infected DCs also showed an enrichment of Gabarap (LC3 family), SQSTM1 a known substrate of autophagy and Rab7, a small GTPase essential for the fusion of autophagosomes to lysosomes (Baba et al., 2019). Galectins including Lgals-3 and Lgals-8 are strongly associated with autophagy; specifically, Lgals-3 binds the Tripartite containing motif protein-16 (TRIM16) to activate autophagy through ATG1/ulk1, we hypothesized that BAdv^{85C5} may induce autophagy through a Lgals-3 and/or Lgals-8 dependent mechanism. Because induction of autophagy enhances the ability of APCs (MΦs; and DCs) to process and present mycobacterial antigen to CD4 T cells (Jagannath et al., 2009), DCs were subjected to siRNA knockdown of Galectins to determine downstream effects. BAdv^{85C5} infected DCs and MΦs were subjected to siRNA vs. Lgal-3 and Lgals-8 and siRNA vs. Lgals-3 for HAdv^{85C5} infected DCs since the later did not express Lgals-8 (FIG. 5B). FIGS. 5C-D illustrate that Galectin knockdown reduced the antigen presentation by both BAdv^{85C5} and HAdv^{85C5} infected DCs and MΦs consistent with their role in regulating autophagy. We recall here that the wild type Adv modulates autophagy through a Nedd4.2 pathway reducing antigen presentation (Montespan et al., 2017). We suggest that BAdv^{85C5} may augment autophagy and antigen presentation through induction of Lgals-3; although, additional studies are required to determine if Lgals-8 synergizes to activate autophagy.

Autophagy-mediated delivery of mycobacteria to lysosomes results in their degradation by lysosomal proteases like cathepsins, lipases, and glycosidases. Indeed, we demonstrated earlier that CTSD cleaves Ag85B in mouse MΦs to generate the p25 epitope, which is then rapidly presented to CD4 T cells in vitro (Singh et al., 2006). Cathepsins not only digest proteins but also help to load peptides into the groove of MHC-II and CTSS and CTSL play a pivotal role (Katunuma et al., 2003; Lee et al., 2006). DC transcriptome studies indicated that BAdv^{85C5} enhanced the expression of multiple cathepsins (FIG. 2F). FIG. 5E shows the qPCR validation of increased cathepsin expression by BAdv^{85C5} DCs compared to HAdv^{85C5} infected DCs. During antigen processing and presentation CTSB, CTSS and CTSL seem to play a major role compared to CTSA, CTSK and CTSZ (Katunuma et al., 2003). Indeed, western blot studies indicated a better expression of CTSB, CTSS and CTSL by BAdv^{85C5} DCs (FIG. 5F). To determine whether cathepsins mediate processing of virus encoded antigens, wt-DCs and ATG7KO-DCs were pharmacologically blocked using pan-specific cathepsin inhibitors, E64 and N-acetyl-L-leucyl-L-leucyl-L-methional (NALLM), and CTSL inhibitor, calpeptin, followed by infection with BAdv^{85C5} or HAdv^{85C5} and antigen presentation. All three cathepsin inhibitors reduced antigen presentation in wt-DCs, whereas, only the pan-inhibitor E64 had an inhibitory effect on BAdv^{85C5} or HAdv^{85C5} infected ATG7KO-DCs (FIG. 5G). These data indicate that BAdv^{85C5} increases the immunogenicity of DCs through cathepsin-dependent antigen processing and presentation.

### BAdv^{85C5} Mucosal Vaccine Protects Mice Against Aerosolized Mtb Either on Its Own or as a Booster Following BCG Vaccine

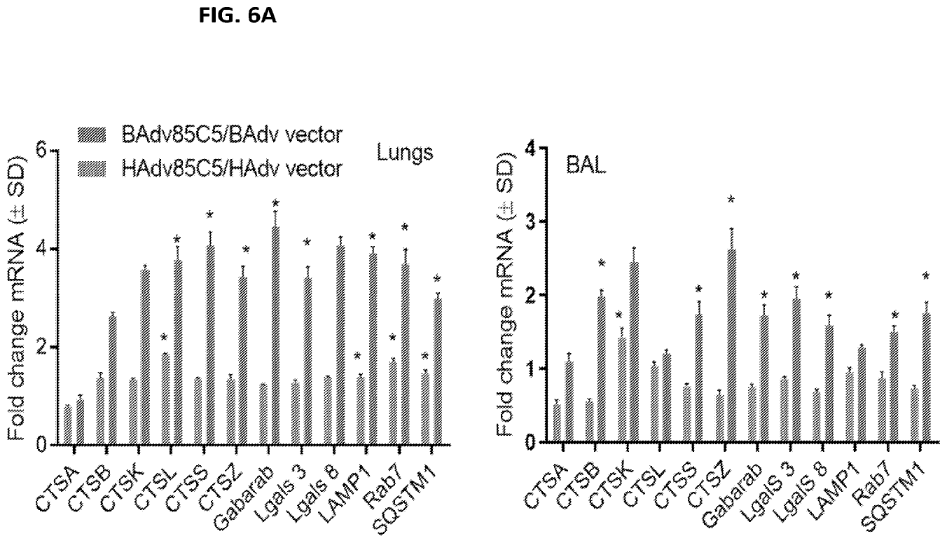

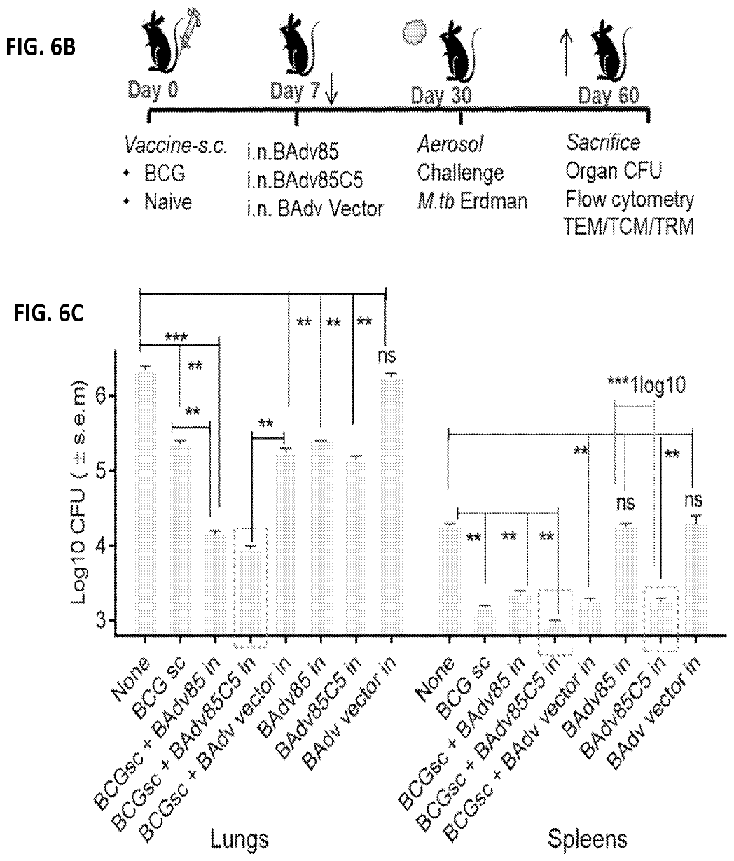

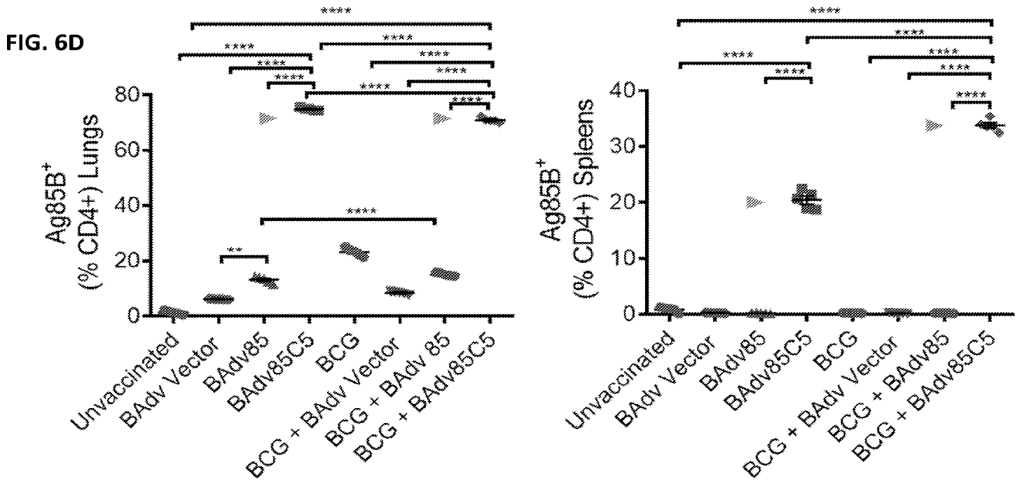

Ex vivo studies indicated that BAdv^{85C5} significantly enhanced the immunogenicity of the DCs in comparison with HAdv^{85C5} vaccine (FIGS. 4-5). Nonetheless, we sought to determine whether BAdv^{85C5} enhanced in vivo immunogenicity. Age and sex matched C57BL/6 mice were inoculated intranasally with vaccines or empty vectors, at 3 days post-inoculation, animals were euthanized, and qPCR analysis of bronchoalveolar (BAL) cells and lungs were performed. FIG. 6A illustrates that BAdv^{85C5} markedly enhanced mRNA transcripts for a panel of genes associated with the mechanisms of antigen processing and presentation compared to the HAdv^{85C5}.

Inbred C57BL/6 show a ‘developing immune system’ akin to the neonatal immune system (Huggins et al., 2019). Thus, C57BL/6 mice are a frequently used model to evaluate the efficacy of BCG and related vaccines. Mice (male and female; 4-6-week-old) were mock-inoculated or vaccinated subcutaneously with BCG, and at 7 days post-immunization, animals received a single intranasal booster vaccination with BAdv⁸⁵, BAdv^{85C5} or BAdv vector control (FIG. 6B). Three weeks later, mice were challenged with aerosolized Mtb Erdman, and at 4 weeks post-challenge, mice were sacrificed, and the lungs and spleen were collected for Mtb bacterial counts and T cell profiles. Compared to the BAdv vector, BAdv^{85C5} induced a robust protection against Mtb as a booster following the primary BCG vaccination, reducing the lung Mtb burden by >1.4 log 10 and spleen load by ˜0.8 log 10 (FIG. 6C). BAdv⁸⁵ also generated a comparable decrease in Mtb CFUs in the lungs and spleen. However, when given alone through intranasal route, only the autophagy-inducing BAdv^{85C5} dramatically reduced the spleen CFUs by ≥1.0 log 10. To confirm the specificity of the Ag85B-p25 immunogenic epitope, the lungs- or spleen-derived T cells were stained using Ag85B-specific tetramer (NIH core, Emory University, USA) and analyzed using flow cytometry. BAdv^{85C5} induced a stronger expansion of Ag85B-p25-specific CD4 T cells in the lungs and spleen, consistent with the ability of the Ag85B-p25 epitope to protect mice against tuberculosis as reported by us and others using mice (FIG. 6D) (Florido et al., 2018; Khan et al., 2019). We note here that BAdv^{85C5} induced a marked expansion of tetramer positive CD4 T cells compared to mice vaccinated using recombinant BCG^{85C5} vaccinated and Mtb challenged mice in our recent study (Khan et al., 2019). This augmentation can be explained by the immunodominance of the p25 epitope and its ability to activate naïve T cells towards Th1 pathway (Tamura et al., 2004). Notably, unlike BCG^{85C5} given intradermally in our recent study, BAdv^{85C5} was given intranasally enabling a robust activation of lung APCs to induce tetramer positive cells. Supporting this observation, co-expression of the AIP-C5 peptide in BAdv^{85C5} led to a robust tetramer positive CD4 T cell response in the lungs compared to the BAdv⁸⁵ vaccine (FIG. 6D). Together, these data suggest that even when given as a single dose of BAdv^{85C5} expressing one major antigenic epitope of Mtb (Ag85B), it serves as a robust mucosal vaccine. Addition of AIP-C5 leads to a dramatic increase in its ability to expand Ag85-p25-specific CD4 T cells (FIG. 6D).

### BAdv^{85C5} Booster Induces Stronger Cytokine Positive T Cells and Effector and Memory T Cells in BCG Vaccinated Mice

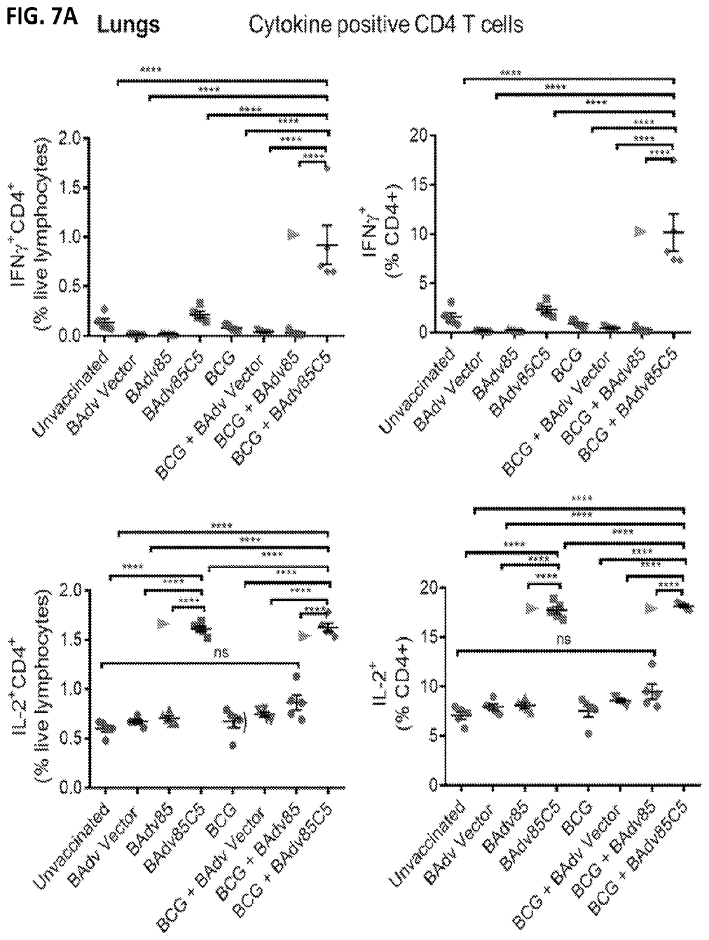

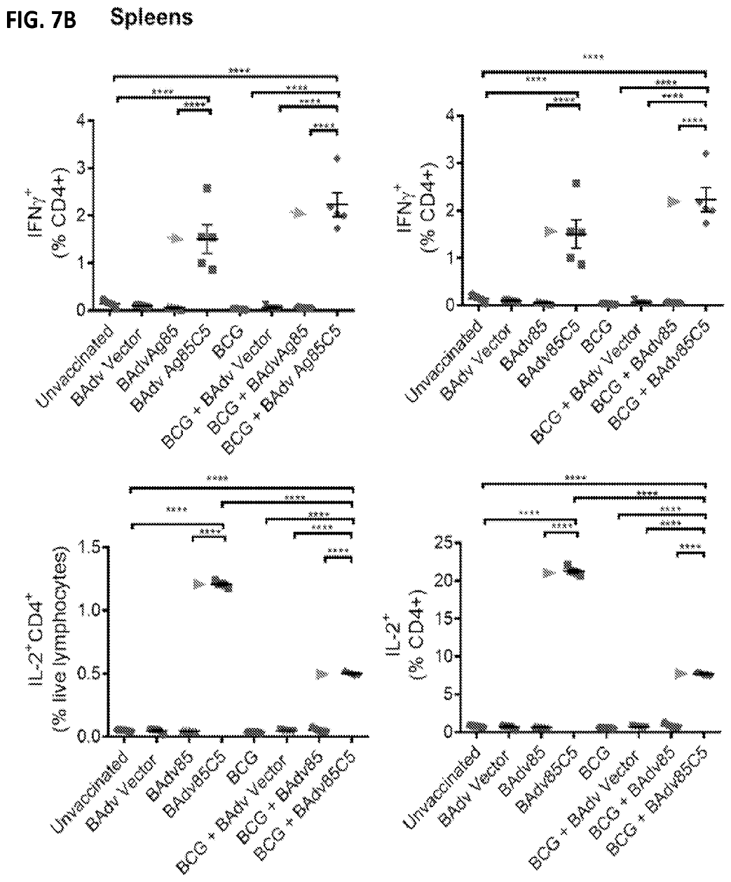

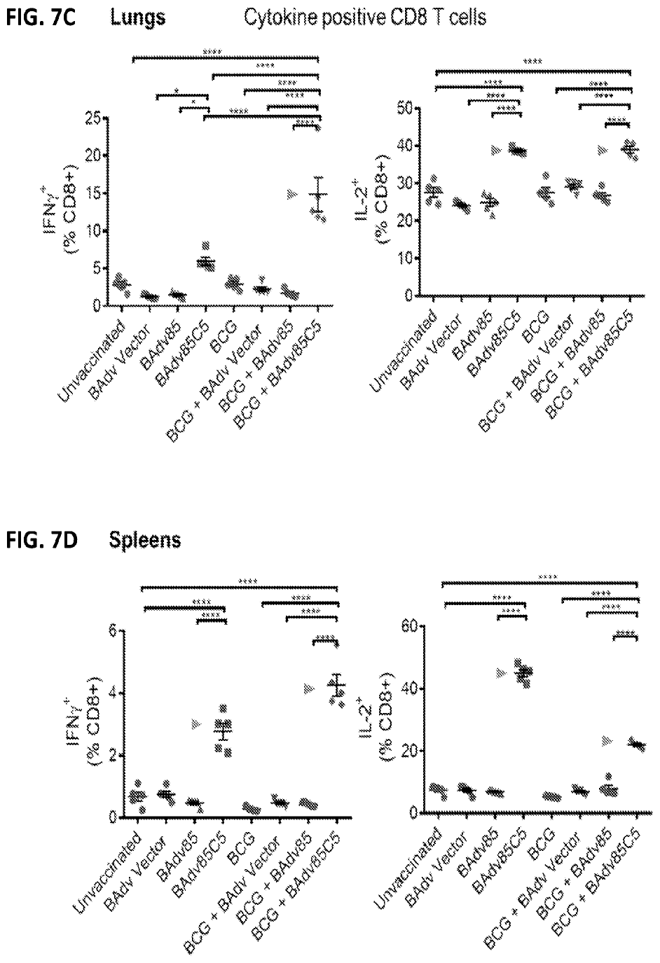

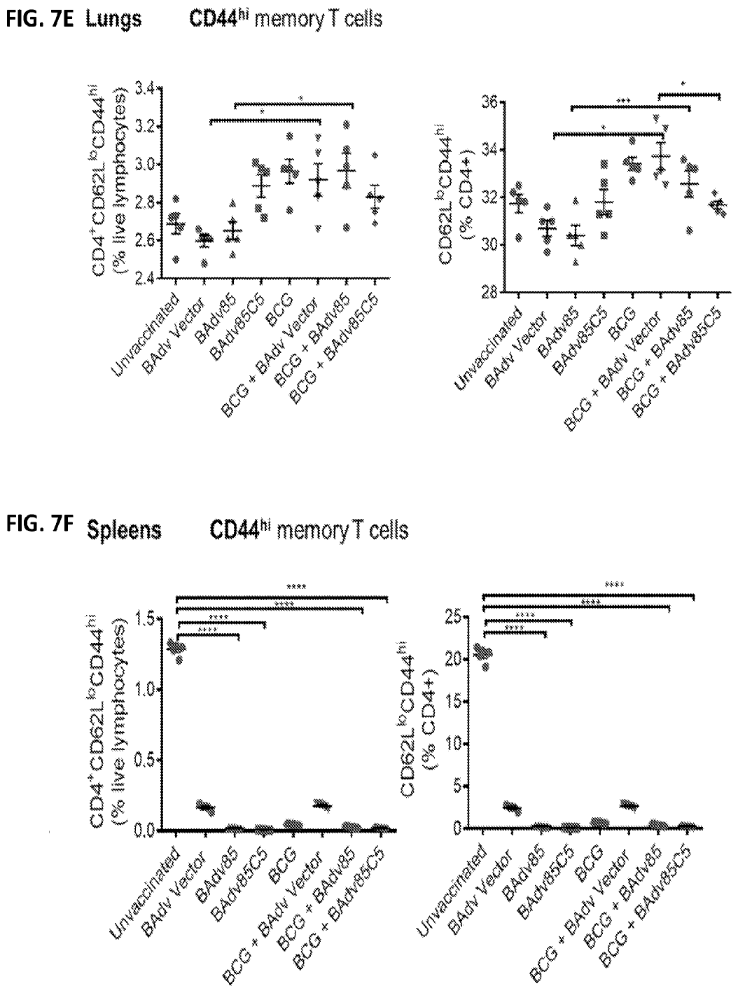

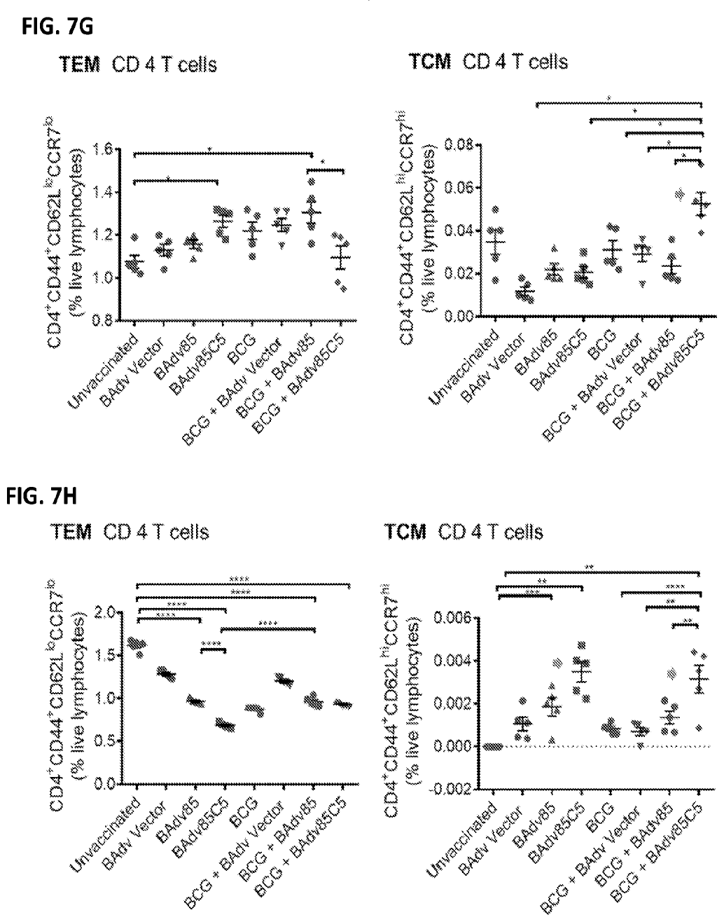

T_{H}1 immunity mediating cytokines like IFN-γ and IL-2 play a major defensive role against tuberculosis in mice and humans (Kaveh et al., 2011). FIGS. 7A-7D illustrate that BAdv^{85C5} vaccine significantly enhanced IFN-γ and IL-2 expressing CD4 and CD8 T cells compared to either BAdv⁸⁵ or BAdv vector. It is important to note here that even when given as an intranasal vaccine, BAdv⁸⁵ induced an expansion of IFN-γ⁺ and IL-2⁺ T cells in both lungs and spleens, suggesting that it can elicit immune responses when given as a nasal vaccine to both children and adults irrespective of BCG vaccination.

It is known that respiratory infections induce an effector T cell response (T_{EM}), which enables an immediate containment of infection, followed by their transition into a long-lasting central memory T cell response (T_{CM}). In many viral infection models and LCMV (lymphocytic choriomeningitis virus) infection of mice, T_{CM} cells mediate long-term immunity (Wherry et al., 2003; Jabbari and Harty, 2006; Gray et al., 2018). Since the lungs are not lymphoid organs, T_{EM} and Tem are thought to arise in the lymph nodes and home back to the lungs for containment of infection recruited by the chemokines secreted from the Mtb-infected MΦs. For example, chemokine receptors CCR2, CXCR5, CCR5 and CXCR6 enhance lung infiltration with TH1 type immune cells, and CCR2-deficient mice are highly susceptible to tuberculosis (Hoft et al., 2019; Peters et al., 2001). FIGS. 7E-7H demonstrate that both BAdv⁸⁵ and BAdv^{85C5} induced an expansion of T_{EM} cells in the lungs and spleens of the BCG-vaccinated mice, although only BAdv^{85C5} induced a significant T_{CM} response. We recall here that BCG induces a robust T_{EM} response but a reduced Tem response in mice (Orme, 2010). Therefore, we propose that using BAdv^{85C5} as a booster in BCG-vaccinated mice can augment long-lived memory T cells.

BAdv^{85C5} Booster Induces a Better Expansion of Lung Resident Memory T (T_{RM}) Cells in BCG-Vaccinated Mice After Mtb Challenge

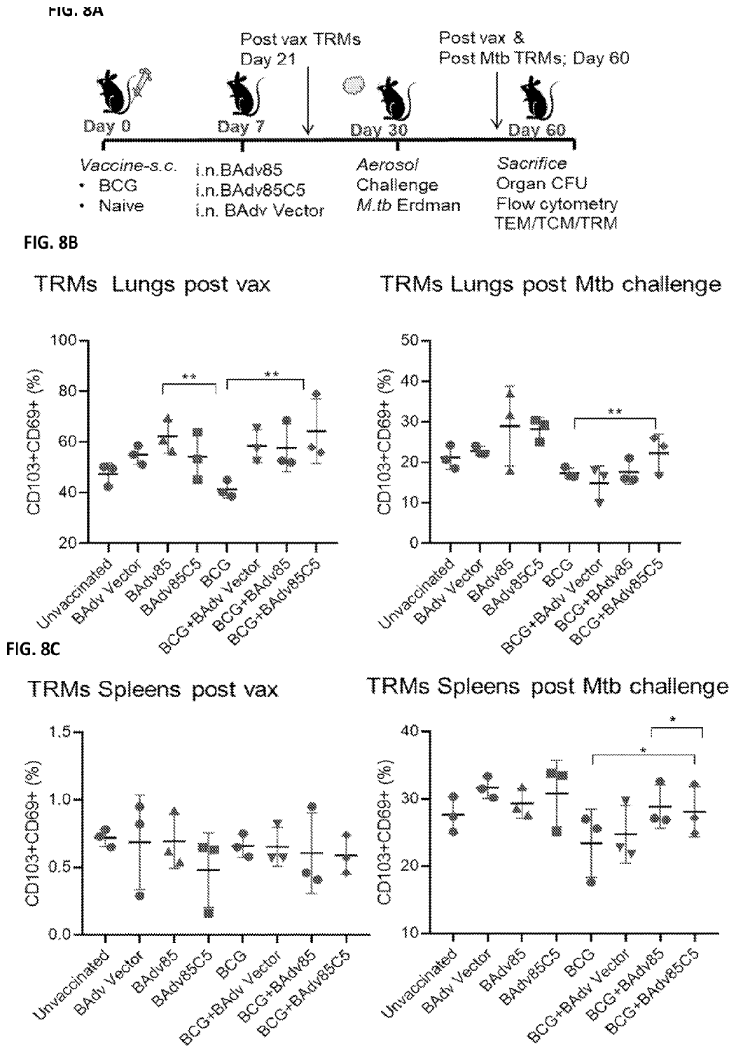

The T_{RM} cells are thought to play a major role in defense against airborne infections (Kumar et al., 2017; Ogongo et al., 2019). The special phenotype of CD4 and CD8 T_{RM} cells are present in the lungs, and recent studies indicate that both human infants and neonatal mice show a physiologically reduced expansion of T_{RM} cells after influenza infection (Zens et al., 2017). Use of an adjuvant with a peptide vaccine augmented lung T_{RM} responses (Thompson et al., 2019). BAdv^{85C5} induced a better T_{RM} response in the lungs of BCG-vaccinated and boosted mice compared to the BCG alone group before and after challenge with Mtb (FIG. 8). It also had a better effect on the expansion of T_{RM} cells in the spleens in the BCG vaccinated, boosted and Mtb challenged group. Because both BAdv⁸⁵ and BAdv^{85C5} expanded T_{RM} cells even when given alone, we suggest that they can generate protection against tuberculosis at the lungs and can be given either to infants or adults at risk.

HAdv-based tuberculosis vaccines have been evaluated in mice (Hoft et al., 2012). In spite of supporting data from the mouse and macaque models, human studies have not been encouraging (Rodo et al., 2019; Darrah et al., 2019). We, therefore, sought to develop an alternative approach using the novel BAdv vaccine platform.

To address pre-existing HAdv vector immunity which could affect vaccine efficacy in humans, we have demonstrated that BAdv vector-based vaccines are equally effective even in the presence of exceptionally high levels of pre-existing HAdv vector immunity (Singh et al., 2008). Besides, BAdv3 internalization is independent of the HAdv5 receptors [Coxackievirus-Adv receptor (CAR) and αvβ3 or αvβ5 integrin] (Bangari et al., 2005a), but utilizes α(2,3)-linked as well as α(2.6)-linked sialic acid as major receptors (Li et al., 2009). Pre-existing HAd-neutralizing antibodies in humans do not cross-neutralize BAdv3 (Bangari et al., 2005b) and HAdv-specific immune response does not cross react with BAdv (Sharma et al., 2010b). Notably, unlike HAdv5, BAdv3 is a strong inducer of TLR4 and does not deplete Kupffer cells of liver (Sharma et al., 2010a), whereas the Kupffer cell depletion with HAdv5 is the main reason for a faster vector depletion from the host. Biodistribution study with a BAdv3 vector showed that it efficiently transduces the heart, kidney, lung, liver and spleen, and the vector persists for a longer duration compared to a HAdv5 vector especially in the heart, kidney and lung in a mouse model (Sharma et al., 2009). Significantly, sequential administration of HAdv5 and BAdv3 vectors overcomes vector immunity in an immunocompetent mouse model of breast cancer (Tandon et al., 2012) and BAdv3 and HAdv5 vector genomes show similar persistence in human and nonhuman cell lines (Sharma et al., 2009). Thus, BAdv3 vectors offer an attractive alternative to HAdv vectors for effectively immunizing individuals with high levels of pre-existing HAdv immunity with safety similar to HAdv5 vectors. Due to the usage of sialic acid as the primary receptor for BAdv, we believe that this vector-based vaccine platform should be ideal for intranasal immunization (Sayedahmed et al., 2018).

This study demonstrates a striking finding that the Mtb Ag85B-p25 epitope expressed using BAdv nasal vaccine platform in combination with AIP-C5 generates a significant protection against Mtb in mice. Our previous studies illustrated that the processing and presentation of mycobacterial antigens by APC's are critical components of vaccine-mediated anti-tuberculosis immunity (Jagannath and Bakhru, 2012; Jagannath et al., 2009). Whereas, BCG and wt-Mtb evade phagolysosome fusion, BCG over-expressing Ag85B (BCG^{85B}), Ag85B plus AIP-C5 (BCG^{85BC5}) or sapM/fbpA gene knockouts of Mtb (ΔfbpAΔsapM-Mtb) were delivered to the lysosomes of mouse APCs generating peptides for activation of CD4 T cells ex vivo (Singh et al., 2006; Saikolappan et al., 2011; Khan et al., 2019). Both BCG^{85B} and BCG^{85BC5} induced robust activation of Ag85-p25 epitope-specific CD4 T cells in mice after vaccination and challenge with Mtb (Jagannath et al., 2009; Khan et al., 2019). However, recombinant BCG vaccines cannot be given to infants through the nasal route due to the risk of lung inflammation. Thus, protection of lungs in neonates following the primary BCG vaccination was our main goal.

Since DCs play a pivotal role during vaccination, we first evaluated gene expression in BAdv^{85C5} infected DCs in comparison with HAdv^{85C5}. Interestingly, BAdv^{85C5} induced a significant up-regulation of multiple genes involved in the sorting of vaccine containing endosomes to lysosomes and genes regulating antigen processing (FIG. 2F). These included, Galectins (Lagals-3, Lgals-8), endosome trafficking proteins (Gabarap, Rab7, LAMPI, LAMP2, CD63, CD53), antigen presentation molecules (B2m, H2-D1, Cathepsins) suggesting that BAdv^{85C5} positively regulates the ability of DCs to present antigen to T cells which is a key event during the expansion of helper T cell responses.

Mechanistic studies were performed to understand the efficacy of BAdv^{85C5} vaccine. Previous studies show that Lgals-8 plays a role during wt-HAdv modulation of autophagy reducing antigen presentation in human epithelial cells (Montespan et al., 2017), whereas, HAdv infection down regulated Lgals-3 (Trinh et al., 2013). Paradoxically, Galectins participate at various levels autophagy; whereas. Lgals-8 inhibits mTOR inducing autophagy (Jia et al., 2018), and Lgals-3 regulate lysosome stability through lysophagy (Yao et al., 2020; Jia et al., 2020). Indeed, our initial studies using BAdv^{85C5} and HAdv^{85C5} showed a reduced antigen presentation to CD4 T cells when ATG7KO-DCs were used (FIG. 4A-4B). Inhibition of autophagy in human MΦs also reduced antigen presentation. Notably, HAdv⁸⁵ infected DCs did present antigen, although the addition of AIP-C5 resulting in HAdv^{85C5} infected significantly increases its ability to present antigen. Therefore, co-expression of AIP-C5 with the Ag85B-p25 epitope not only bypassed the ability of HAdv to interfere with autophagy but enhanced the ability of BAdv⁸⁵ infected DCs. We further demonstrate that BAdv^{85C5} induces a robust expression of both Lgals-3 and Lgals-8 compared to HAdv^{85C5} (FIG. 5F) which facilitated antigen presentation, since siRNA blockade led to a down regulation of antigen presentation (FIGS. 5C-5D).

Consistent with our previous observation that induction of autophagy enhances MHC-II dependent antigen presentation by mycobacteria infected DCs (Jagannath et al., 2009), BAdv⁸⁵-infected DCs up-regulated several genes which likely facilitated an autophagy-dependent antigen presentation. Thus, LAMP2 participates during chaperone-mediated autophagy when soluble proteins of cytosol are internalized into lysosomes (Tekirdag and Cuervo, 2018; Wang et al., 2016), and Gabarap (LC3 family) and Rab7 are involved during selective autophagy (Khaminets et al., 2015) and autophagolysosome fusion, respectively. Because, multiple immune-regulatory genes were expressed at lower levels following infection of ATG7KO-DCs with BAdv^{85C5} compared to wt-DCs, the signaling pathways which deliver BAdv⁸⁵ autophagolysosomes need additional investigations. However, we found a novel cathepsin-dependent mechanism through which BAdv⁸⁵ infected DCs show increased immunogenicity compared to HAdv⁸⁵.

BAdv^{85C5} enhanced the cathepsin gene and protein expression compared to HAdv^{85C5} (FIG. 5E-F). Cathepsins play a major role during antigenic peptide production within lysosomes facilitated by their acidic pH. For example, CTSD cleaves mycobacterial Ag85B to produce the p25 epitope which is then presented to CD4 T cells (Singh et al., 2006), whereas, CTSB, CTSS and CTSL play a pivotal role in digesting antigens and loading microbial peptides into the groove of MHC-II (Katunuma et al., 2003). Validating the gene expression profiles, a pharmacological blockade of cathepsins led to a near abrogation of antigen presentation by BAdv^{85C5} and HAdv^{85C5} in infected DCs, using a broad spectrum cathepsin inhibitor E64 (FIG. 5G). Earlier studies an intriguing effect of cathepsins. CTSG induced the killing of Mtb within THP-1 MΦs, whereas a blockade of CTSB, CTSS or CTSL enhanced the growth of Mtb in human MΦs (Rivera-Marrero et al., 2004; Pires et al., 2016). To determine whether BAdv^{85C5}-induced lysosomal proteases (FIG. 5F-5G) help to degrade mycobacteria, mouse CD14⁺ MΦs were infected with BAdv or HAdv vectors or vaccines followed by a coinfection with Mtb. We found that both BAdv⁸⁵ and HAdv^{85C5} exerted a bactericidal effect suggesting an intriguing concept that our new generation vaccines may have a therapeutic effect.

These data together indicate that BAdv^{85C5} is better than HAdv^{85C5} in enhancing lysosomal production of the Ag85B-p25 epitope thereby boosting the immunogenicity of DCs. We recall here that the mammalian target of rapamycin, mTOR, which is a major negative regulator of autophagy, controls the accumulation of DCs in the lungs and governs the programing of their metabolomes (Sinclair et al., 2017). Since BAdv^{85C5} induces positive changes in DCs mainly through autophagy pathway (FIGS. 2-5), we propose that it can alter the lung DC populations for better antigen-mediated activation of T cells, particularly when given through the nasal route. We also note here that the Modified Vaccinia Ankara (MVA) vaccine induced apoptosis of DCs in the draining lymph nodes (Guzman et al., 2012), suggesting that it may be one reason for the failure of MVA85A to protect against tuberculosis in children. In our studies, BAdv^{85C5} was rapidly internalized into DCs (FIG. 2), facilitating a sustained and elevated antigen presentation and DCs remained fully viable during the in vitro assays (FIG. 4E).

Consistent with the in vitro immunogenicity data, the BAdv^{85C5} vaccine showed a marked protective effect in mice exposed to Mtb. First, BAdv^{85C5} induced a robust gene expression in the lungs and BAL of mice when given as a single nasal vaccine compared to BAdv⁸⁵ (FIG. 6A). Secondly, the vaccine induced ˜1.4-log reduction of Mtb burden in the lungs compared to BCG alone, when given as a booster following BCG (FIG. 6B-C). For a single dose of a single epitope with a nasal booster vaccine, this appears to be the best level of protection in mice reported for any Adv vaccine for tuberculosis. Besides, a single dose of a nasal vaccine alone reduced the Mtb burden in both the lungs and spleen suggesting that this vaccine strategy can protect adults at risk for tuberculosis.

Immunity against tuberculosis is multifactorial, including TH1-immunity facilitated by cytokine secreting CD4 and CD8 T cells, a variety of other innate T cells, neutrophils, myeloid cells and antibodies. FIG. 7 illustrates that the BAdv^{85C5} vaccine induced an expansion of IFN-γ and IL-2 secreting CD4 and CD8 T cells in both BCG-vaccinated mice and those receiving only the BAdv^{85C5} vaccine. Most tuberculosis vaccines induce memory T cells, which can be classified into an early T_{EM} response followed by a longer-lasting T_{CM} response. Interestingly, BCG vaccine is a poor inducer of T_{CM} response in mice (Orme, 2010); although, recombinant BCG vaccines induce stronger T_{CM} in mice correlating with a better protection against tuberculosis (Vogelzang et al., 2014; Khan et al., 2019). Although the role of Tem response in protecting against primary exposure to Mtb is debated, it appears that T_{CM} cells play a major role in defending against reinfection and reactivation of Mtb. For example, HIV-induced CD4 T cell depletion predisposes the reactivation of Mtb, implying that at least CD4 T_{CM} cells persist in a resting stage among those vaccinated with BCG or exposed to Mtb. These resting T_{CM} cells can rapidly expand into T_{EM} cells to contain Mtb after exposure. In our investigation, BAdv^{85C5} vaccination led to an expansion of both T_{EM} and T_{EM} cells in BCG-vaccinated mice (FIG. 7). Curiously, BAdv^{85C5} given alone expanded T_{CM} cells in the spleen (FIG. 7), indicating that the BAdv^{85C5} vaccine can be used even among adults to activate T_{CM} cells. This is significant because, at least in the mouse model, BCG seems to be a poor inducer of T_{CM} response (Orme, 2010). We, therefore, propose that intradermal BCG vaccination of neonates followed by a nasal booster with BAdv85C5 will stimulate longer-lasting immunity, whereas adults exposed to index cases of tuberculosis can also be protected by the BAdv^{85C5} vaccine when given alone.

BCG revaccination of healthy volunteers reduces skin test conversion, but the impact of this issue to the susceptibility for tuberculosis remains unclear. Because, intranasal BCG is not feasible, we propose that BAdv^{85C5} nasal vaccine will strengthen the lung compartment to resist aerosol infection with Mtb. Supporting this concept, the BAdv^{85C5} booster enhanced the T_{RM} response in the lungs of BCG-vaccinated mice before and after Mtb exposure (FIG. 8). This is significant because human and mouse neonates show a reduced expansion of T_{RM} cells after influenza infection (Zens et al., 2017). Others have suggested that during RSV vaccination, the use of TLR activation may help in overcoming the hypo-responsiveness of T_{RM} cells in the lungs (Zhang et al., 2017). In this context, we recall here that the AIP-C5, activates TLR-2 in both mouse and human APCs inducing autophagy (Khan et al., 2019). Although additional studies are required, we consider that the TLR2-dependent adjuvant effect of AIP-C5 may have contributed to the robust T cells responses in the mouse lungs following BAdv^{85C5} vaccination, and it will likely boost the neonatal lung immunity when given as a nasal vaccine.

In summary, the BAdv vaccine platform enables the expression of an Mtb immunogenic peptide and activates Lgals-3 and Lgals-8 dependent autophagy leading to robust antigen presentation by both mouse DCs and human MΦs resulting in an excellent protection in the mouse model of tuberculosis. Our results suggest that autophagy-mediated antigen presentation through the BAdv vaccine platform expressing multivalent immunogenic proteins of Mtb will assist in designing a more potent mucosal tuberculosis vaccine for use in children and adults.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### CITED REFERENCES

Ahi, Y.S., Bangari, D.S., and Mittal, S.K. (2011). Adenoviral vector immunity: its implications and circumvention strategies. Curr Gene Ther 11, 307-320.
Baba, T., Toth, D.J., Sengupta, N., Kim, Y.J., and Balla, T. (2019). Phosphatidylinositol 4,5-bisphosphate controls Rab7 and PLEKHM1 membrane cycling during autophagosome-lysosome fusion. EMBO J 38, e100312.
Bailey, C.J., Crystal, R.G., and Leopold, P.L. (2003). Association of adenovirus with the microtubule organizing center. J Virol 77, 13275-13287.
Bakhru, P. (2012). Induction of stronger and long lasting vaccine mediated immunity to tuberculosis. In Pathology (Houston: University of Texas Health Sciences Center), pp. 155.
Bakhru, P., Sirisaengtaksin, N., Soudani, E., Mukherjee, S., Khan, A., and Jagannath., C. (2013). BCG vaccine mediated reduction in MHC-II expression of macrophages and dendritic cells can be reversed using activation of Toll-like receptors 7 and 9. Cellular Immunology (2013).
Bangari, D.S., and Mittal, S.K. (2006). Current strategies and future directions for eluding adenoviral vector immunity. Curr Gene Ther 6, 215-226.
Bangari, D.S., Sharma, A., and Mittal, S.K. (2005a). Bovine adenovirus type 3 internalization is independent of primary receptors of human adenovirus type 5 and porcine adenovirus type 3. Biochem Biophys Res Commun 331, 1478-1484.
Bangari, D.S., Shukla, S., and Mittal, S.K. (2005b). Comparative transduction efficiencies of human and nonhuman adenoviral vectors in human, murine, bovine, and porcine cells in culture. Biochem Biophys Res Commun 327, 960-966.
Barouch, D.H., Tomaka, F.L., Wegmann, F., Stich, D.J., Alter, G., Robb, M.L., Michael, N.L., Peter, L., Nkolola, J.P., Borducchi, E.N., et al. (2018). Evaluation of a mosaic HIV-1 vaccine in a multicentre, randomised, double-blind, placebo-controlled, phase ½a clinical trial (APPROACH) and in rhesus monkeys (NHP 13-19). Lancet 392, 232-243.
Cao, W., Liepkalns, J.S., Hassan, A.O., Kamal, R.P., Hofstetter, A.R., Amoah, S., Kim, J.H., Reber, A.J., Stevens, J., Katz, J.M., et al. (2016). A highly immunogenic vaccine against A/H7N9 influenza virus. Vaccine 34, 744-749.
Chauhan, S., Kumar, S., Jain, A., Ponpuak, M., Mudd, M.H., Kimura, T., Choi, S.W., Peters, R., et al. (2016). TRIMs and Galectins Globally Cooperate and TRIM16 and Galectin-3 Co-direct Autophagy in Endomembrane Damage Homeostasis. Dev Cell 39, 13-27.
Chen, M., Hong. M.J., Sun, H., Wang, L., Shi, X., Gilbert, B.E., Corry, D.B., Kheradmand, F., and Wang, J. (2014). Essential role for autophagy in the maintenance of immunological memory against influenza infection. Nat Med 20, 503-510.
Darrah, P.A., DiFazio, R.M., Maiello, P., Gideon, H.P., Myers, A.J., Rodgers, M.A., Hackney, J.A., Lindenstrom, T., Evans, T., et al. (2019). Boosting BCG with proteins or rAd5 does not enhance protection against tuberculosis in rhesus macaques. NPJ Vaccines 4, 21.
Fausther-Bovendo, H., and Kobinger, G.P. (2014). Pre-existing immunity against Ad vectors: humoral, cellular, and innate response. Hum Vaccin Immunother 10, 2875-2884.
Florido, M., Muflihah, H., Lin, L.C.W., Xia, Y., Sierro, F., Palendira, M., Feng, C.G., Bertolino, P., Stambas, J., Triccas, J.A., et al. (2018). Pulmonary immunization with a recombinant influenza A virus vaccine induces lung-resident CD4 (+) memory T cells that are associated with protection against tuberculosis. Mucosal Immunol.
Florido, M., Pillay, R., Gillis, C.M., Xia, Y., Turner, S.J., Triccas, J.A., Stambas, J., and Britton, W.J. (2015). Epitope-specific CD4+, but not CD8+, T-cell responses induced by recombinant influenza A viruses protect against Mycobacterium tuberculosis infection. Eur J Immunol 45, 780-793.
Gengenbacher, M., Nieuwenhuizen, N., Vogelzang, A., Liu, H., Kaiser, P., Schuerer, S., Lazar, D., Wagner, I., Mollenkopf, H.J., and Kaufmann, S.H. (2016). Deletion of nuoG from the Vaccine Candidate Mycobacterium bovis BCG DeltaureC::hly Improves Protection against Tuberculosis. MBio 7.
Gray, J.I., Westerhof, L.M., and Macleod, M.K.L. (2018). The roles of resident, central and effector memory CD4 T-cells in protective immunity following infection or vaccination. Immunology.
Guzman, E., Cubillos-Zapata, C., Cottingham, M.G., Gilbert, S.C., Prentice, H., Charleston, B., and Hope, J.C. (2012). Modified vaccinia virus Ankara-based vaccine vectors induce apoptosis in dendritic cells draining from the skin via both the extrinsic and intrinsic caspase pathways, preventing efficient antigen presentation. J Virol 86, 5452-5466.
Hendrickx. R., Stichling, N., Koelen, J., Kuryk, L., Lipiec, A., and Greber, U.F. (2014). Innate immunity to adenovirus. Hum Gene Ther 25, 265-284.
Hoelscher, M.A., Garg, S., Bangari, D.S., Belser, J.A., Lu, X., Stephenson, I., Bright, R.A., Katz, J.M., Mittal, S.K., and Sambhara, S. (2006). Development of adenoviral-vector-based pandemic influenza vaccine against antigenically distinct human H5N1 strains in mice. Lancet 367, 475-481.
Hoelscher, M.A., Jayashankar, L., Garg, S., Veguilla, V., Lu, X., Singh, N., Katz, J.M., Mittal, S.K., and Sambhara, S. (2007). New pre-pandemic influenza vaccines: an egg- and adjuvant-independent human adenoviral vector strategy induces long-lasting protective immune responses in mice. Clin Pharmacol Ther 82, 665-671.
Hoft, D.F., Blazevic, A., Stanley, J., Landry, B., Sizemore, D., Kpamegan, E., Gearhart, J., Scott, A., Kik, S., Pau, M.G., et al. (2012). A recombinant adenovirus expressing immunodominant TB antigens can significantly enhance BCG-induced human immunity. Vaccine 30, 2098-2108.
Hoft, S.G., Sallin, M.A., Kauffman, K.D., Sakai, S., Ganusov, V.V., and Barber, D.L. (2019). The Rate of CD4 T Cell Entry into the Lungs during Mycobacterium tuberculosis Infection Is Determined by Partial and Opposing Effects of Multiple Chemokine Receptors. Infect Immun 87.
Huggins, M.A., Sjaastad, F.V., Pierson, M., Kucaba, T.A., Swanson, W., Staley, C., Weingarden, A.R., Jensen, I.J., Danahy, D.B., Badovinac, V.P., et al. (2019). Microbial Exposure Enhances Immunity to Pathogens Recognized by TLR2 but Increases Susceptibility to Cytokine Storm through TLR4 Sensitization. Cell Rep 28, 1729-1743 e1725.
Jabbari, A., and Harty, J.T. (2006). Secondary memory CD8+T cells are more protective but slower to acquire a central-memory phenotype. J Exp Med 203, 919-932.
Jagannath, C., and Bakhru, P. (2012). Rapamycin-induced enhancement of vaccine efficacy in mice. Methods Mol Biol 821, 295-303.
Jagannath, C., Lindsey, D.R., Dhandayuthapani, S., Xu, Y., Hunter, R.L., Jr., and Eissa, N.T. (2009). Autophagy enhances the efficacy of BCG vaccine by increasing peptide presentation in mouse dendritic cells. Nat Med 15, 267-276.
Jia, J., Abudu, Y.P., Claude-Taupin, A., Gu, Y., Kumar, S., Choi, S.W., Peters, R., Mudd, M.H., Allers, L., Salemi, M., et al. (2018). Galectins Control mTOR in Response to Endomembrane Damage. Mol Cell 70, 120-135 e128.
Jia, J., Claude-Taupin, A., Gu, Y., Choi, S.W., Peters, R., Bissa, B., Mudd, M.H., Allers, L., Pallikkuth, S., Lidke, K.A., et al. (2020). Galectin-3 Coordinates a Cellular System for Lysosomal Repair and Removal. Dev Cell 52, 69-87 e68.
Katunuma, N., Matsunaga, Y., Himeno, K., and Hayashi, Y. (2003). Insights into the roles of cathepsins in antigen processing and presentation revealed by specific inhibitors. Biol Chem 384, 883-890.
Kaveh, D.A., Bachy, V.S., Hewinson, R.G., and Hogarth, P.J. (2011). Systemic BCG immunization induces persistent lung mucosal multifunctional CD4 T (EM) cells which expand following virulent mycobacterial challenge. PLOS One 6, e21566.
Khaminets, A., Heinrich, T., Mari, M., Grumati, P., Huebner, A.K., Akutsu, M., Liebmann, L., Stolz, A., Nietzsche, S., Koch, N., et al. (2015). Regulation of endoplasmic reticulum turnover by selective autophagy. Nature 522, 354-358.
Khan, A., Bakhru, P., Saikolappan, S., Das, K., Soudani, E., Singh, C.R., Estrella, J.L., Zhang, D., Pasare, C., Ma, Y., et al. (2019). An autophagy-inducing and TLR-2 activating BCG vaccine induces a robust protection against tuberculosis in mice. NPJ Vaccines 4, 34.
Kumar, B.V., Ma, W., Miron, M., Granot, T., Guyer, R.S., Carpenter, D.J., Senda, T., Sun, X., Ho, S.H., Lerner, H., et al. (2017). Human Tissue-Resident Memory T Cells Are Defined by Core Transcriptional and Functional Signatures in Lymphoid and Mucosal Sites. Cell Rep 20, 2921-2934.
Ledgerwood, J.E., DeZure, A.D., Stanley, D.A., Coates, E.E., Novik, L., Enama, M.E., Berkowitz, N.M., Hu, Z., Joshi, G., Ploquin, A., et al. (2017). Chimpanzee Adenovirus Vector Ebola Vaccine. N Engl J Med 376, 928-938.
Lee, H.K., Mattei, L.M., Steinberg, B.E., Alberts, P., Lee, Y.H., Chervonsky, A., Mizushima, N., Grinstein, S., and Iwasaki, A. (2010). In vivo requirement for Atg5 in antigen presentation by dendritic cells. Immunity 32, 227-239.
Lee, J.A., Sinkovits, R.S., Mock, D., Rab, E.L., Cai, J., Yang, P., Saunders, B., Hsueh, R.C., Choi, S., Subramaniam, S., et al. (2006). Components of the antigen processing and presentation pathway revealed by gene expression microarray analysis following B cell antigen receptor (BCR) stimulation. BMC Bioinformatics 7, 237.
Leopold, P.L., Ferris, B., Grinberg, I., Worgall, S., Hackett, N.R., and Crystal, R.G. (1998). Fluorescent virions: dynamic tracking of the pathway of adenoviral gene transfer vectors in living cells. Hum Gene Ther 9, 367-378.
Li, F.Y., Wang, S.F., Bernardes, E.S., and Liu, F.T. (2020). Galectins in Host Defense Against Microbial Infections. Adv Exp Med Biol 1204, 141-167.
Li, X., Bangari, D.S., Sharma, A., and Mittal, S.K. (2009). Bovine adenovirus serotype 3 utilizes sialic acid as a cellular receptor for virus entry. Virology 392, 162-168.
Maier, O., Marvin, S.A., Wodrich, H., Campbell, E.M., and Wiethoff, C.M. (2012). Spatiotemporal dynamics of adenovirus membrane rupture and endosomal escape. J Virol 86, 10821-10828.
Montespan, C., Marvin, S.A., Austin, S., Burrage, A.M., Roger, B., Rayne, F., Faure, M., Campell, E.M., Schneider, C., Reimer, R., et al. (2017). Multi-layered control of Galectin-8 mediated autophagy during adenovirus cell entry through a conserved PPxY motif in the viral capsid. PLOS Pathog 13, e1006217.
Munz, C. (2015). Of LAP, CUPS, and DRibbles-Unconventional Use of Autophagy Proteins for MHC Restricted Antigen Presentation. Front Immunol 6, 200.
Nishida, Y., Arakawa, S., Fujitani, K., Yamaguchi, H., Mizuta, T., Kanaseki, T., Komatsu, M., Otsu, K., Tsujimoto, Y., and Shimizu, S. (2009). Discovery of Atg5/Atg7-independent alternative macroautophagy. Nature 461, 654-658.
Ogongo, P., Porterfield, J.Z., and Leslie, A. (2019). Lung Tissue Resident Memory T-Cells in the Immune Response to Mycobacterium tuberculosis. Front Immunol 10, 992.
Orme, I.M. (2010). The Achilles heel of BCG. Tuberculosis (Edinb) 90, 329-332.
Perez-Montesinos, G., Lopez-Ortega, O., Piedra-Reyes, J., Bonifaz, L.C., and Moreno, J. (2017). Dynamic Changes in the Intracellular Association of Selected Rab Small GTPases with MHC Class II and DM during Dendritic Cell Maturation. Front Immunol 8, 340.
Peters, W., Scott, H.M., Chambers, H.F., Flynn, J.L., Charo, I.F., and Ernst, J.D. (2001). Chemokine receptor 2 serves an early and essential role in resistance to Mycobacterium tuberculosis. Proc Natl Acad Sci USA 98, 7958-7963.
Pires, D., Marques, J., Pombo, J.P., Carmo, N., Bettencourt, P., Neyrolles, O., Lugo-Villarino, G., and Anes, E. (2016). Role of Cathepsins in Mycobacterium tuberculosis Survival in Human Macrophages. Sci Rep 6, 32247.
Ramachandra, L., Noss, E., Boom, W.H., and Harding, C.V. (1999). Phagocytic processing of antigens for presentation by class II major histocompatibility complex molecules. Cell Microbiol 1, 205-214.
Ramachandra, L., Noss, E., Boom, W.H., and Harding, C.V. (2001). Processing of Mycobacterium tuberculosis antigen 85B involves intraphagosomal formation of peptide-major histocompatibility complex II complexes and is inhibited by live bacilli that decrease phagosome maturation. J Exp Med 194, 1421-1432.
Ramzan, M., Ali, S.M., Malik, A., Zaka-ur-Rab, Z., and Shahab, T. (2009). Frequency of HIV infection amongst children with disseminated tuberculosis and tuberculous meningitis in Aligarh (North India)-a low HIV prevalence area. J Coll Physicians Surg Pak 19, 566-569.
Rivera-Marrero, C.A., Stewart, J., Shafer, W.M., and Roman, J. (2004). The down-regulation of cathepsin G in THP-1 monocytes after infection with Mycobacterium tuberculosis is associated with increased intracellular survival of bacilli. Infect Immun 72, 5712-5721.
Rodo, M.J., Rozot, V., Nemes, E., Dintwe, O., Hatherill, M., Little, F., and Scriba, T.J. (2019). A comparison of antigen-specific T cell responses induced by six novel tuberculosis vaccine candidates. PLOS Pathog 15, e1007643.
Saikolappan, S., Estrella, J., Khan, A., Smitha, K., Jagannath, C., and Dhandayuthapani, S. (2011). The deltafbpA/deltasapM double knocout mutant derived from Mycobacterium tuberculosis H37Rv is attenuated and highly immunogenic in macrophages (under revision). PLOSone.
Saini, N.K., Baena, A., Ng, T.W., Venkataswamy, M.M., Kennedy, S.C., Kunnath-Velayudhan, S., Carreno, L.J., Xu, J., Chan, J., Larsen, M.H., et al. (2016). Suppression of autophagy and antigen presentation by Mycobacterium tuberculosis PE_PGRS47. Nat Microbiol 1, 16133.
Santosuosso, M., McCormick, S., Zhang, X., Zganiacz, A., and Xing, Z. (2006). Intranasal boosting with an adenovirus-vectored vaccine markedly enhances protection by parenteral Mycobacterium bovis BCG immunization against pulmonary tuberculosis. Infect Immun 74, 4634-4643.
Saso, A., and Kampmann, B. (2017). Vaccine responses in newborns. Semin Immunopathol 39, 627-642.
Sayedahmed, E.E., Hassan, A.O., Kumari, R., Cao, W., Gangappa, S., York, I., Sambhara, S., and Mittal, S.K. (2018). A bovine adenoviral vector-based H5N1 influenza-vaccine provides enhanced immunogenicity and protection at a significantly low dose. Mol Ther Methods Clin Dev 10, 210-222.
Schaaf, M.B., Keulers, T.G., Vooijs, M.A., and Rouschop, K.M. (2016). LC3/GABARAP family proteins: autophagy-(un) related functions. FASEB J 30, 3961-3978.
Segura, E., and Amigorena, S. (2015). Cross-Presentation in Mouse and Human Dendritic Cells. Adv Immunol 127, 1-31.
Sharma, A., Bangari, D.S., Tandon, M., Hogenesch, H., and Mittal, S.K. (2010a). Evaluation of innate immunity and vector toxicity following inoculation of bovine, porcine or human adenoviral vectors in a mouse model. Virus Res 153, 134-142.
Sharma, A., Bangari, D.S., Tandon, M., Pandey, A., HogenEsch, H., and Mittal, S.K. (2009). Comparative analysis of vector biodistribution, persistence and gene expression following intravenous delivery of bovine, porcine and human adenoviral vectors in a mouse model. Virology 386, 44-54.
Sharma, A., Tandon, M., Ahi, Y.S., Bangari, D.S., Vemulapalli, R., and Mittal, S.K. (2010b). Evaluation of Cross-Reactive Cell-Mediated Immune Responses among Human, Bovine and Porcine Adenoviruses. Gene Ther 17, 634-642.
Sharma, P.K., Dmitriev, I.P., Kashentseva, E.A., Raes, G., Li, L., Kim, S.W., Lu, Z.H., Arbeit, J.M., Fleming, T.P., Kaliberov, S.A., et al. (2018). Development of an adenovirus vector vaccine platform for targeting dendritic cells. Cancer Gene Ther 25, 27-38.
Sinclair, C., Bommakanti, G., Gardinassi, L., Loebbermann, J., Johnson, M.J., Hakimpour, P., Hagan, T., Benitez, L., Todor, A., Machiah, D., et al. (2017). mTOR regulates metabolic adaptation of APCs in the lung and controls the outcome of allergic inflammation. Science 357, 1014-1021.
Singh, C.R., Bakhru, P., Khan, A., Li, Q.B., and Jagannath, C. (2011). Cutting Edge: Nicastrin and Related Components of gamma-Secretase Generate a Peptide Epitope Facilitating Immune Recognition of Intracellular Mycobacteria, through MHC Class II—Dependent Priming of T Cells. J Immunol 187, 5495-5499.
Singh, C.R., Moulton, R.A., Armitige, L. Y., Bidani, A., Snuggs, M., Dhandayuthapani, S., Hunter, R.L., and Jagannath, C. (2006). Processing and presentation of a mycobacterial antigen 85B epitope by murine macrophages is dependent on the phagosomal acquisition of vacuolar proton ATPase and in situ activation of cathepsin D. J Immunol 177, 3250-3259.
Singh, N., Pandey, A., Jayashankar, L., and Mittal, S.K. (2008). Bovine adenoviral vector-based H5N1 influenza vaccine overcomes exceptionally high levels of pre-existing immunity against human adenovirus. Mol Ther 16, 965-971.
Smaill, F., Jeyanathan, M., Smieja, M., Medina, M.F., Thanthrige-Don, N., Zganiacz, A., Yin, C., Heriazon, A., Damjanovic, D., Puri, L., et al. (2013). A human type 5 adenovirus-based tuberculosis vaccine induces robust T cell responses in humans despite preexisting anti-adenovirus immunity. Sci Transl Med 5, 205ra134.
Soualhine, H., Deghmane, A.E., Sun, J., Mak, K., Talal, A., Av-Gay, Y., and Hmama, Z. (2007). Mycobacterium bovis Bacillus Calmette-Guerin secreting active cathepsin S stimulates expression of mature MHC class II molecules and antigen presentation in human macrophages. J Immunol 179, 5137-5145.
Tameris, M.D., Hatherill, M., Landry, B.S., Scriba, T.J., Snowden, M.A., Lockhart, S., Shea, J.E., McClain, J.B., Hussey, G.D., Hanekom, W.A., et al. (2013). Safety and efficacy of MVA85A, a new tuberculosis vaccine, in infants previously vaccinated with BCG: a randomised, placebo-controlled phase 2b trial. Lancet 381, 1021-1028.
Tamura, T., Ariga, H., Kinashi, T., Uehara, S., Kikuchi, T., Nakada, M., Tokunaga, T., Xu, W., Kariyone, A., Saito, T., et al. (2004). The role of antigenic peptide in CD4+ T helper phenotype development in a T cell receptor transgenic model. Int Immunol 16, 1691-1699.
Tandon, M., Sharma, A., Vemula, S.V., Bangari, D.S., and Mittal, S.K. (2012). Sequential administration of bovine and human adenovirus vectors to overcome vector immunity in an immunocompetent mouse model of breast cancer. Virus Res 163, 202-211.
Tekirdag, K., and Cuervo, A.M. (2018). Chaperone-mediated autophagy and endosomal microautophagy: Joint by a chaperone. J Biol Chem 293, 5414-5424.
Thompson, E.A., Darrah, P.A., Foulds, K.E., Hoffer, E., Caffrey-Carr, A., Norenstedt, S., Perbeck, L., Seder, R.A., Kedl, R.M., and Lore, K. (2019). Monocytes Acquire the Ability to Prime Tissue-Resident T Cells via IL-10-Mediated TGF-beta Release. Cell Rep 28, 1127-1135 e1124.
Thurston, T.L., Wandel, M.P., von Muhlinen, N., Foeglein, A., and Randow, F. (2012). Galectin 8 targets damaged vesicles for autophagy to defend cells against bacterial invasion. Nature 482, 414-418.
Trinh, H.V., Grossmann, J., Gehrig, P., Roschitzki, B., Schlapbach, R., Greber, U.F., and Hemmi, S. (2013). iTRAQ-Based and Label-Free Proteomics Approaches for Studies of Human Adenovirus Infections. Int J Proteomics 2013, 581862.
Van Kampen, K.R., Shi, Z., Gao, P., Zhang, J., Foster, K.W., Chen, D.T., Marks, D., Elmets, C.A., and Tang, D.C. (2005). Safety and immunogenicity of adenovirus-vectored nasal and epicutaneous influenza vaccines in humans. Vaccine 23, 1029-1036.
van Zyl-Smit, R.N., Esmail, A., Bateman, M.E., Dawson, R., Goldin, J., van Rikxoort, E., Douoguih, M., Pau, M.G., Sadoff, J.C., McClain, J.B., et al. (2017). Safety and Immunogenicity of Adenovirus 35 Tuberculosis Vaccine Candidate in Adults with Active or Previous Tuberculosis. A Randomized Trial. Am J Respir Crit Care Med 195, 1171-1180.
Vemula, S.V., and Mittal, S.K. (2010). Production of adenovirus vectors and their use as a delivery system for influenza vaccines. Expert Opin Biol Ther 10, 1469-1487.
Vogelzang, A., Perdomo, C., Zedler, U., Kuhlmann, S., Hurwitz, R., Gengenbacher, M., and Kaufmann, S.H. (2014). Central memory CD4+ T cells are responsible for the recombinant Bacillus Calmette-Guerin DeltaureC::hly vaccine's superior protection against tuberculosis. J Infect Dis 210, 1928-1937.
Wang, B., Chen, Z., Yu, F., Chen, Q., Tian, Y., Ma, S., Wang, T., and Liu, X. (2016). Hsp90 regulates autophagy and plays a role in cancer therapy. Tumour Biol 37, 1-6.
Weng, I.C., Chen, H.L., Lo, T.H., Lin, W.H., Chen, H.Y., Hsu, D.K., and Liu, F.T. (2018). Cytosolic galectin-3 and-8 regulate antibacterial autophagy through differential recognition of host glycans on damaged phagosomes. Glycobiology 28, 392-405.
Wherry, E.J., Teichgraber, V., Becker, T.C., Masopust, D., Kaech, S.M., Antia, R., von Andrian, U.H., and Ahmed, R. (2003). Lineage relationship and protective immunity of memory CD8 T cell subsets. Nat Immunol 4, 225-234.
Xing, Z., McFarland, C.T., Sallenave, J.M., Izzo, A., Wang, J., and McMurray, D.N. (2009). Intranasal mucosal boosting with an adenovirus-vectored vaccine markedly enhances the protection of BCG-primed guinea pigs against pulmonary tuberculosis. PLOS One 4, e5856.
Yao, R.Q., Ren, C., Xia, Z.F., and Yao, Y.M. (2020). Organelle-specific autophagy in inflammatory diseases: a potential therapeutic target underlying the quality control of multiple organelles. Autophagy, 1-17.
Yu, J.C., Khodadadi, H., Malik, A., Davidson, B., Salles, E., Bhatia, J., Hale, V.L., and Baban, B. (2018). Innate Immunity of Neonates and Infants. Front Immunol 9, 1759.
Zens, K.D., Chen, J.K., Guyer, R.S., Wu, F.L., Cvetkovski, F., Miron, M., and Farber, D.L. (2017). Reduced generation of lung tissue-resident memory T cells during infancy. J Exp Med 214, 2915-2932.
Zhang, L., Li, H., Hai, Y., Yin, W., Li, W., Zheng, B., Du, X., Li, N., Zhang, Z., Deng, Y., et al. (2017). CpG in Combination with an Inhibitor of Notch Signaling Suppresses Formalin-Inactivated Respiratory Syncytial Virus-Enhanced Airway Hyperresponsiveness and Inflammation by Inhibiting Th17 Memory Responses and Promoting Tissue-Resident Memory Cells in Lungs. J Virol 91.
Zhu, J., Huang, X., and Yang, Y. (2007). Innate immune response to adenoviral vectors is mediated by both Toll-like receptor-dependent and-independent pathways. J Virol 81, 3170-3180.

What is claimed is:

1. A recombinant adenovirus vector comprising a heterologous DNA segment encoding mycobacterial Ag85B-p25 epitope (SEQ ID NO: 1), mycobacterial Ag85B-p25 epitope fusion of autophagy-inducing peptide-C5 (SEQ ID NO: 2), or a substantially homologous functional fragment thereof.

2. The recombinant adenovirus vector of claim 1, wherein the heterologous DNA segment encoding mycobacterial Ag85B-p25 epitope has a SEQ ID NO: 3.

3. The recombinant adenovirus vector of claim 1, wherein the vector is a replication defective human adenovirus vector or a bovine adenovirus vector.

4. (canceled)

5. (canceled)

6. (canceled)

7. (canceled)

8. (canceled)

9. (canceled)

10. The recombinant adenovirus vector of claim 1, wherein the heterologous DNA segment is operationally fused in an expression cassette with an autophagy-inducing peptide such as AIP-C5.

11. A method of therapeutically or prophylactically immunizing a subject comprising administering to the subject a vaccine formulation of recombinant adenovirus vector having a heterologous DNA segment encoding mycobacterial Ag85B-p25 epitope (SEQ ID NO: 1), mycobacterial Ag85B-p25 epitope fusion of autophagy-inducing peptide-C5 (SEQ ID NO: 2), or a substantially homologous functional fragment thereof.

12. The method of claim 11, wherein the heterologous DNA segment encoding mycobacterial Ag85B-p25 epitope has a SEQ ID NO: 3.

13. The method of claim 11, wherein the recombinant adenovirus vector is a replication-defective human adenovirus vector or a bovine adenovirus vector.

14. The method of claim 11, wherein the vaccine formulation is useful as an effective vaccine for protection from infections by a microorganism.

15. The method of claim 14, wherein the microorganism is a fungus, a virus, or a bacteria.

16. The method of claim 14, wherein the microorganism is Mycobacterium tuberculosis (Mtb).

17. The method of claim 11, wherein the vaccine formulation is useful as an effective mucosal vaccine.

18. The method of claim 11, wherein the vaccine formulation is useful as an effective vaccine delivered nasally.

19. The method of claim 11, wherein the vaccine formulation is an effective vaccine for tuberculosis.

20. The method of claim 11, wherein the vaccine formulation is useful as an effective vaccine by way of infecting dendritic cells (DCs) and thereby upregulating a transcriptome of genes regulating antigen processing.

21. The method of claim 11, wherein the heterologous DNA segment is operationally fused in an expression cassette with an autophagy-inducing peptide such as AIP-C5.

22. The method according to claim 11, wherein the subject is a human being or an animal.

23. A pharmaceutical composition for therapeutically or prophylactically immunizing a subject comprising a recombinant adenovirus vector having a heterologous DNA segment encoding mycobacterial Ag85B-p25 epitope (SEQ ID NO: 1), mycobacterial Ag85B-p25 epitope fusion of autophagy-inducing peptide-C5 (SEQ ID NO: 2), or a substantially homologous functional fragment thereof, together with one or more pharmaceutically acceptable carriers, diluents, or excipients.

24. The pharmaceutical composition for therapeutically or prophylactically immunizing a subject of claim 23, wherein the heterologous DNA segment encoding mycobacterial Ag85B-p25 epitope having has a SEQ ID NO: 3.

25. The pharmaceutical composition according to claim 23, wherein the recombinant adenovirus vector is a replication-defective human adenovirus vector, a bovine adenovirus vector or any other adenovirus vector.

26. The pharmaceutical composition according to claim 23, wherein the recombinant adenovirus vector is useful as an effective vaccine for protection from infections by a microorganism.

27. The pharmaceutical composition according to claim 26, wherein the microorganism is a fungus, a virus, or a bacteria.

28. The pharmaceutical composition according to claim 26, wherein the microorganism is Mycobacterium tuberculosis (Mtb).

29. The pharmaceutical composition according to claim 23, wherein the recombinant adenovirus vector is useful as an effective mucosal vaccine.

30. The pharmaceutical composition according to claim 23, wherein the recombinant adenovirus vector is useful as an effective vaccine delivered nasally.

31. The pharmaceutical composition according to claim 23, wherein the recombinant adenovirus vector is an effective vaccine for tuberculosis.

32. The pharmaceutical composition according to claim 23, wherein said vector is useful as an effective vaccine by way of infecting dendritic cells (DCs) and thereby upregulating a transcriptome of genes regulating antigen processing.

33. The pharmaceutical composition according to claim 23, wherein the heterologous DNA segment is operationally fused in an expression cassette with an autophagy-inducing peptide such as AIP-C5.

34. The pharmaceutical composition according to claim 23, wherein the subject is a human being or an animal.

35. The recombinant adenovirus vector of claim 1, wherein the heterologous DNA segment encoding mycobacterial Ag85B-p25 epitope fusion of autophagy-inducing peptide-C5 has a SEQ ID NO: 4.

36. The method of claim 11, wherein the heterologous DNA segment encoding mycobacterial Ag85B-p25 epitope fusion of autophagy-inducing peptide-C5 has a SEQ ID NO: 4.

37. The method of claim 20, wherein the transcriptome of genes comprises CTSA, CTSB, CTSK, CTSL, CTSS, CTSZ, Gabarap, Lgals-3, Lgals-8, LAMP1, Rab7, and SQSTM1.

38. The method of claim 20, wherein the transcriptome of genes comprises B2M, CD53, CD63, CD68, Clec4e, H2D1, Hsp9a, LILRB4, and LAMP2.

39. The pharmaceutical composition for therapeutically or prophylactically immunizing a subject of claim 23, wherein the heterologous DNA segment encoding mycobacterial Ag85B-p25 epitope fusion of autophagy-inducing peptide-C5 has a SEQ ID NO: 4.

40. The method of claim 32, wherein the transcriptome of genes comprises CTSA, CTSB, CTSK, CTSL, CTSS, CTSZ, Gabarap, Lgals-3, Lgals-8, LNAM1, Rab7, and SQSMTM1.

41. The method of claim 32, wherein the transcriptome of genes comprises B2M, CD53, CD63, CD68, zvlrv4e, H2D1, Hsp9a, LILRB4, and LAMP2.
